(19) United States
(12) Patent Application Publication
(10) Pub. No.: US 2013/0041265 A1
(43) Pub. Date: Feb. 14, 2013

(54) METHODS AND APPARATUS FOR INTRODUCING CELLS AT A TISSUE SITE

(71) Applicants: Ron Sostek, Newton, MA (US); David Green, Dover, MA (US)

(72) Inventors: Ron Sostek, Newton, MA (US); David Green, Dover, MA (US)

(21) Appl. No.: 13/497,436

(22) PCT Filed: Sep. 21, 2010
(86) PCT No.: PCT/US10/02595

§ 371 Date: Oct. 29, 2012

Related U.S. Application Data
(60) Provisional application No. 61/244,434, filed on Sep. 21, 2009.
(60) Provisional application No. 61/298,414, filed on Jan. 26, 2010.

(51) Int. Cl. A61M 5/00; A61B 6/00; A61M 5/44

(52) U.S. Cl. 600/473; 604/93.01; 604/113

(57) ABSTRACT

The present invention relates to methods and devices for maintaining cellular viability and function for therapeutic purposes. The invention provides methods and devices for maintaining the proliferative and developmental potential of cellular preparations by protecting the from physical and physiological damage during storage, preparation, and delivery to a site (e.g., a tissue site). The invention also provides methods and devices for evaluating tissues and organs, and selecting appropriate sites for cellular delivery.

### RELATED APPLICATIONS

This application claims priority under 35 U.S.C. §119(e) from U.S. Provisional Application Ser. No. 61/244,434 with a filing date of Sep. 21, 2009 and entitled “Methods and Apparatus for Introducing Cells at a Tissue Site” and U.S. Provisional Application Ser. No. 61/298,414 with a filing date of Jan. 26, 2010 and entitled “Methods and Apparatus for Introducing Cells at a Tissue Site” the contents of both of which are incorporated herein in their entirety by reference.

### FIELD OF THE INVENTION

Aspects of the present invention relate to methods and apparatus for introducing cells to a target site, e.g., a tissue site in a subject. In particular, aspects of the invention relate to providing functional cells for therapeutic applications and delivering them to subjects at particular sites to treat one or more diseases or disorders.

### BACKGROUND OF INVENTION

Cell-based therapies have been developed to treat a range of medical conditions that are associated with cellular loss or damage. For example, neurodegenerative disorders, cardiovascular conditions (including infarcts), and other conditions associated with cell death or injury can be treated by injecting appropriate cells to replace one or more damaged cell types at a tissue site in a subject. Current methods have been optimized to maintain the viability of cells that are being injected using standard syringes or injectors, e.g, syringes or injectors designed for drugs and/or analyte delivery. However, there is a need for improved cellular delivery devices and methods to support the further development of cell-based therapies.

### SUMMARY

Aspects of the invention relate to cell introduction devices that are adapted to protect and deliver a viable and functional cellular mass to a tissue site. Unlike drugs, cells cannot be highly concentrated for delivery without addressing certain factors, e.g., aggregation, temperature, nutritional, metabolic by-products, etc. According to the invention, over-concentration or other mishandling of a cellular mass or its surrounding environment may disrupt or otherwise deactivate one or more desirable physiological (e.g., metabolic, nutritional, communication, migration, contractility) or pharmacological activities even if the cells remain viable. Aspects of the invention relate to effective delivery devices and methods adapted to protect a viable mass of cells before, during, and/or after the delivery process to a target site.

Further aspects of the invention relate to systems, devices and methods for improved cellular delivery. In some embodiments, the systems, devices and methods support cell-based therapies by addressing factors associated with cell homeostasis, such as, for example, physiological, metabolic, anatomical (e.g., mass and shape), respiratory, environmental, nutritional, and cellular communication factors. In some embodiments, systems and devices are provided that are designed and configured to preserve and/or activate cells properly; related methods are provided in some embodiments.

In some embodiments, Applicants have recognized that the success of cell-based therapies can be significantly enhanced by providing a delivery technology that is adapted to monitor and/or maintain an appropriate physiological environment for the cells throughout one or more phases of the process of introducing the cells (e.g., by injection, e.g., as an aerosol) to a target site. In some embodiments, Applicants have recognized that cell-based therapies can be significantly enhanced by providing cell preparation and/or storage technologies that ensure cells are in a condition suitable for delivery. In some embodiments, the cell preparation and storage technologies enable cell freezing and thawing in a manner that promotes or ensures cell viability during injection. Applicants also have recognized that the evaluation and selection of one or more appropriate target sites significantly enhances the survival and function of the cells being introduced (e.g., injected). In some embodiments, methods and devices of the invention can help maintain the functional potential (e.g., potential for proliferation and/or differentiation) of a therapeutic cell (e.g., a stem or progenitor cell) that may otherwise be lost even though the cells remain viable using traditional techniques.

In some embodiments, aspects of the invention relate to a system and method for introducing cells at a target (e.g., tissue) site. For example, differentiated or undifferentiated stem or other cells may be introduced on or in a tissue, such as heart tissue, brain or spinal cord tissue, lung tissue, liver tissue, pancreatic tissue, other solid organ tissues, and other tissue sites. The cells may be introduced at the tissue site for a variety of different purposes, such as to grow and replace dead or dying cells at the tissue site, to reconnect nerve cell connections severed by accident or other cause, and so on. For example, in the case of heart attack, heart tissue in one or more localized areas may die or suffer severe injury due to lack of blood flow. To help repair the damage, stem cells may be introduced at and/or near the site of damage so that the cells may grow into the damaged area and effectively replace the damaged tissue. The cells are introduced below the tissue surface in one or more areas, e.g., tissue sites, so that the cells may grow and function as other heart tissue. In some embodiments, systems and methods of the invention may be used to deliver viable and functional cells to other sites such as matrices that are useful for growing tissue or organs ex vivo.

Aspects of the invention relate to devices and methods that may be used with any suitable cell type for therapeutic and/or research purposes. For example, devices and methods provided by some aspects of the invention may be used to process and/or inject various types of pluripotent, multipotent, or oligopotent stem cells, or their differentiated progeny, for example, for a therapeutic or research purpose. Examples of cells that can be injected using a device or method provided by some aspects of this invention include embryonic stem cells (ESCs), adult stem cells (ASCs), and induced pluripotent stem cells (iPSCs) and differentiated cells derived from any of these stem cell types. Examples of cells that can be injected using a method or device provided by some aspects of this invention for a therapeutic purpose include, but are not limited to, neural and neuronal stem and precursor cells and their differentiated progeny (e.g., neurons, oligodendrocytes, astrocytes, ependymal cells, radial glia, Schwann cells, or satellite cells), cardiac stem cells and their differentiated progeny (e.g., cardiomyocytes), mesenchymal stem or progenitor cells and their differentiated progeny (e.g., osteoblasts, chondrocytes, and adipocytes), endothelial stem or progenitor cells, stem cell-derived islet cells, and stem cell-derived hepatocytes. However, other cell types also may be used, as aspects of the invention are not limited in this respect. Cells may be derived from any species (e.g., human, primate, other mammals, or other species) that is suitable for the application being considered. In some embodiments, aspects of the invention may used for personalized medicine or research by using cells that are derived from a subject (e.g., human patient) being treated. In some embodiments, cells are derived from plants.

Aspects of the invention relate to methods and devices for protecting cells before, during, and/or after introduction to a target site within a recipient. Such devices are different from conventional introduction devices (e.g., syringes with needle-like injectors used in drug delivery), because cellular delivery devices may be adapted for handling cells before the delivery (filtration, temperature, metabolic monitoring and control), during the delivery, and/or after the delivery. Accordingly, in some embodiments a cellular introduction device may have unique characteristics to handle the consequences of cells having a vacuole and a cell mass that does not dissolve in solution (unlike drugs that can be dissolved). Due to the properties of vacuoles and the cell mass, cells can be fractured (e.g., by excessive force, osmotic concentration variables, defrosting variables, pH changes, etc., or any combination thereof), damaged, and/or deactivated (e.g., resulting in a loss of certain physiological and/or pharmacokinetic properties). This functional degradation can occur during every phase of cellular preparation and delivery, and even after delivery. Cells have metabolic requirements that make it important to appropriately manage temperature, respiration and metabolic variables and products (e.g., waste, communication chemicals, e.g., cytokines). In some embodiments, one or more of these is monitored, adjusted, and in some cases eliminated (e.g., waste toxins). In some embodiments, careful control of the cellular environment before, during, and after injection reduces the amount of injured and damaged cellular mass (e.g., the mass of cells being damaged after the injection from tissue fiber resistance that damages cells before they can migrate.

In some embodiments, an injector is also physically configured to avoid or reduce cellular damage. For examples, injectors may be designed to minimize destruction at or surrounding the site of injection. In some embodiments, injectors may have one or more of the following structures: a frit or filter at the end working end of the injector, an adsorptive device at the end of (or anywhere in) the path, a coating with an absorbing material, a filter (e.g., for cellular debris, toxins, or other contaminants) to prevent contaminants from being injected, a cross-sectional shape and/or area that reduces or minimizes tissue damage. It should be appreciated that a filter may include one or more suitable filtering mechanism (e.g., chemical, ionic, absorption, etc.).

Accordingly, in some embodiments, devices and methods include one or more features for maintaining a viable cellular environment prior to introduction (e.g., by maintaining appropriate temperature, oxygen, and pH levels). In some embodiments, devices and methods are provided for protecting cells from physical and/or chemical damage during the introduction process (e.g., to protect the cells from excessive pressure or shear stress during injection). In some embodiments, devices and methods are provided to protect cells from physical, chemical, and/or biological harm (e.g., due to physical trauma and/or host response) after introduction into a recipient (e.g., by minimizing recipient tissue damage and/or providing support for the cells after introduction). Devices and methods described herein provide significant advantages over current techniques for injecting cells into tissue (e.g., muscle) that are similar to those used for injecting drugs into a blood vessel using a simple syringe. According to aspects of the invention, the failure to protect cells that are being introduced into a recipient may account for the observed high levels of cell death during cellular transplantation (up to 95% of injected cells die according to reports in the literature). This results in low yields and limited medical beneficial results.

It should be appreciated that systems, devices, and methods of the invention may be used to repair organ or tissue damage in any multi-cellular organism, for example, in animals, vertebrates, mammals, or other multi-cellular subjects. In some embodiments, systems, devices, and methods of the invention may be used to influence, train, modify, or otherwise alter the behavior of existing cells at a target site. In some embodiments, aspects of the invention are used to treat domestic and/or agricultural animals. In some embodiments, aspects of the invention are used to treat humans (e.g., human patients having one or more tissue or organ defects, for example, due to disease and/or injury). In some embodiments, subjects (e.g., human patients) may be treated one or more times according to aspects of the invention. In some embodiments, subjects may be monitored after treatment, e.g., to evaluate the progression of a disease or disorder and/or to evaluate the effectiveness of a treatment. In some embodiments, cells may be injected into a subject at a tissue site using a device or system of the invention. In some embodiments, a device or system of the invention may be implantable (e.g., including a working end, a pump, a controller, a power source, and/or one or more additional or alternative components). Accordingly, in some embodiments a device or system of the invention may be implanted at a tissue site in a subject in need thereof. In some embodiments, cells may be introduced into plants.

Cells may be introduced at a tissue site by a working end of a cell introduction device, which may be the end of a needle-like member or other tube-shaped structure with one or more openings (e.g., at the end and/or on one or more sides of the working end) from which cells may be released. In some embodiments, a cell introduction device may have a single working end. However, in some embodiments, a device may have a plurality of working ends (e.g., a plurality of needle-like elements or penetrating structures may be arranged in linear arrays, spatial arrays, single tubes or other geometric shapes to maximizes tissue penetration and cell delivery).

It should be appreciated that a tube-shaped structure may be a cylindrical structure with a circular cross-section in some embodiments. However, in some embodiments the tube-shape structure is an elongated member that may have any suitable shape in cross-section (e.g., oval, triangular, square, rectangular, pentagonal, hexagonal, any other regular or irregular shape, or any combination thereof. In some embodiments, a tube-shaped structure may include one or more tapered and/or flared segments and/or ends.

It should be appreciated that injectors or components thereof (e.g., the tube-shaped structure, or needle, etc.) may be made of any suitable material including, but not limited to, one or more of the following: a metal, carbon, a ceramic, a polymer, a plastic, a glass, or any other suitable material. In some embodiments, tube-shaped structures (e.g., a needle) may comprise or consist of carbon nanotubes. Flexible connectors (e.g., tubes) may be made of any suitable plastic, rubber, polymer, or other flexible material.

It should be appreciated that the shape and material of the working end (and any other part of the injector) may be adapted for the intended use. For example, a longer flexible tube-shaped structure with high conformational compliance may be suited for injection into the brain, whereas a shorter and more rigid tube-shaped structure may be suited for a harder organ (e.g., the kidney): A rectangular or triangular cross-section may be useful for organs (such as the kidney) that have a lattice-like structure in order to penetrate and possibly promote the formation of a tissue crack or fissure into which a cellular solution may be injected.

Cells may be provided through the one or more openings in a variety of different ways, such as by pressure, diffusion, and other mechanisms. In one embodiment, the cell introduction device may include a syringe like device having a working end extending from a reservoir of any suitable shape and size (e.g., the reservoir may be tubular or any other suitable shape that has a sufficient internal volume and configuration to contain and deliver a cellular preparation) and plunger or other cell displacing technology. Cells may be delivered using any suitable technique (e.g., cell displacement, pressure, osmotic, dialysis, electric charge, etc., or any combination thereof) that can be applied to the reservoir to force fluid containing cells (cell fluid or cell material) in the reservoir from an opening in at the working end (e.g., at the end of the needle-like-like device). A distal end of the device (the working end) may be inserted into tissue, and the plunger or displacement technology moved to force fluid, including desired cells, from the opening at the distal end of the device (e.g., the needle-like device). Although a syringe arrangement is one illustrative embodiment that may be used with various aspects of the invention, the cell introduction device may have other arrangements described in more detail below. For example, a mechanical pump may be used to cause fluid flow in a working end of a cell introduction device, and the operation of the pump may be controlled based on one or more sensed parameters, such as pressure of fluid in the working end, a flow rate of cells at the working end, a total volume of cells released from the working end, shear stress on cells, and/or other parameters. It should be appreciated that any suitable cell delivery technique may be used (e.g., cell displacement, pressure, osmotic, dialysis, electric charge, iontophoresis, electro-osmosis, etc., or any combination thereof) as aspects of the invention are not limited in this respect. In some embodiments, a device includes two or more working ends for injecting cells (e.g., in linear arrays and/or spatial arrays), and cells may be delivered through the different working ends using one or a combination of different delivery techniques.

A cell introduction device or system may be designed to reduce physical trauma associated with shear stress and/or severe pressure gradients during the introduction process. In some embodiments, a controller may be used to regulate the rate and/or pressures used to inject cells into a tissue site. In some embodiments, the physical configuration of an injector may be designed to avoid features that create shear stress and/or undesirable pressure gradients. In some embodiments, the physical configuration of an injector may be designed to include features that reduce shear stress and/or undesirable pressure gradients.

In some embodiments, the injection system may have a holding device or include synchronization technology that can facilitate injection into moving organs (e.g., heart, lungs, etc.). These holding and/or synchronization configurations facilitate the synchronization of the syringe-like device or other injector into the moving tissue by synchronizing and reducing the differential frequencies presented by the moving tissue and the delivery mechanism.

A cell introduction device or system may be designed to reduce chemical or biological or physical trauma associated with inappropriate growth or maintenance conditions (e.g., temperature, pH, oxygen levels, waste products, cellular debris, shear force, communication chemicals (e.g., cytokines), etc.) during the introduction process.

In some aspects of the invention, printers are provided for printing compositions comprising biological cells.

Accordingly, aspects of the invention relate to systems, devices, and components thereof (e.g., syringes, arrays, etc.) that have features adapted for protecting the function and viability of therapeutic cell preparations during storage, defrosting, immediately prior to injection, during injection, and/or after injection at a site (e.g., a tissue site). It should be appreciated that any of the components described herein may be sterilized prior to use in a subject. Any suitable sterilization technique may be used (e.g., irradiation, chemical treatment, heat, etc., or any combination thereof). These and other aspects are described in more detail herein.

### BRIEF DESCRIPTION OF DRAWINGS

The accompanying drawings, are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates a non-limiting embodiment of a cell introduction device;

FIG. 2 illustrates a non-limiting embodiment of a cell introduction device connected with a pump and controller;

FIG. 3 illustrates a non-limiting embodiment of a cell introduction device having a plurality of working ends;

FIG. 4A illustrates a non-limiting embodiment of a device that includes a movable member that may be positioned relative to the working end to prevent the working end from being inserted into underlying tissue beyond a predetermined depth in accordance with some embodiments of the invention;

FIG. 4B illustrates non-limiting embodiments of devices that include a plurality of working ends arranged in an array, and further illustrates a device positioned such that the working ends are disposed within a tissue;

FIG. 5 illustrates an anchoring device that may be used to support and/or guide a cell introducing device at a tissue site in accordance with some embodiments of the invention;

FIG. 6 depicts an illustrative map showing injection flow path in which the intensities correspond to temperatures;

FIG. 7 illustrates non-limiting embodiments of cell introduction devices having a angled working ends;

FIG. 8 illustrates a non-limiting embodiment of an injector array with a vacuum for attaching to a tissue;

FIG. 9A illustrates a non-limiting embodiment of a cell introduction device configured with a pressure transducer;

FIG. 9B illustrates a non-limiting embodiment of a cell introduction device configured with a pressure transducer;

FIG. 10 illustrates a non-limiting embodiment of a defrost system in which a support device (e.g., a chip) containing cells may be stored in a frozen state;

FIG. 11 illustrates a non-limiting embodiment a tool that may be used to identify tissue sites;

FIG. 12 illustrates a non-limiting embodiment of a support device also referred to as a containment module and corresponding injecting device;

FIG. 13 illustrates a non-limiting embodiment of a heart that is being evaluated to identify its pattern of spatial vibrational and heat distributions in accordance with some embodiments of the invention;

FIG. 14 illustrates a non-limiting embodiment of a cylindrical rolling electrode;

FIG. 15A illustrates a non-limiting example of an insertable probe comprising an elongated insertable member attached to a support member.

FIG. 15B illustrates a non-limiting embodiment of a device having an array of insertable members attached to a first surface of a support member thereby forming a patch;

FIG. 16 illustrates a non-limiting embodiment of a device comprising insertable elements that are designed as energy deflectors/concentrators; and

FIG. 17 illustrates a non-limiting embodiment of a cell delivery system comprising a vortex mixer for mixing cells and an integrated controller.

### DETAILED DESCRIPTION

Aspects of the invention relate to cell introduction devices that are adapted to protect and deliver a viable and functional cellular mass (or suspension) to a target site, e.g., tissue site. In some embodiments, aspects of the invention are directed to methods and devices for preparing and/or delivering a cellular preparation to a tissue site in a subject (e.g., a patient being treated with a cell-based therapy). Methods and devices are configured to provide one or more features that help preserve cellular function before, during, and/or after introduction (e.g., by injection) into a target site, e.g., tissue, scaffold. Unlike drugs, cells cannot be highly concentrated for delivery without deleterious effects unless proper care, as described in certain aspects of the invention, is employed. In some embodiments, methods and devices are provided that enable delivery of relatively high concentrations of cells. According to the invention, over-concentration or other mishandling of a cellular mass may disrupt or otherwise deactivate one or more desirable physiological and/or functional activities (e.g., a desirable pharmacological activity) even if the cells remain viable. Aspects of the invention relate to effective delivery devices and methods adapted to protect cells or membrane-bound structures (e.g., cells or artificial membrane-bound structures greater than about 2 microns in diameter) before, during, and/or after the delivery process to a tissue site.

Techniques that may be useful to protect the functionality and/or viability of cells in a therapeutic application include devices and methods for i) protecting the cells and the physiological environment of the cells (e.g., metabolic conditions, respiratory state, communication state, chemical concentration, oxygen levels, temperature, nutrient levels, waste product levels, etc., or any combination thereof) prior to and during injection, including, but not limited to, needle size and shape, filters, components for regulating temperature and/or oxygen levels, or any combination thereof; ii) adjusting the pressure and volume of the fluid being injected; iii) providing needles that are adapted for injection into a tissue site, including, but not limited to, the shape of the injection needle(s), the number and size of the needles, the configuration of the needles, the presence of collars to limit injection depth, or any combination thereof; iv) providing supports to assist in the injection process, including, but not limited to the use of a needle support that can be attached to the site of injection using a vacuum or other technique, the use of a support or guide for an injection device, or any combination thereof; v) monitoring the physiology of the cells prior to and during injection, for example, using infrared or other detection technology; vi) evaluating the tissue site of injection, for example, using infrared, vibration patterns, or other technology; and vii) integrated systems and devices for performing one or more techniques described herein. It should be appreciated that in some embodiments, techniques described herein may include one or more databases of information relating to one or more parameters being monitored and/or adjusted for a cellular injection process. In some embodiments, techniques that may be useful to protect the functionality and/or viability of cells in a therapeutic application may comprise devices and methods for providing nutritional supplementation to the cells.

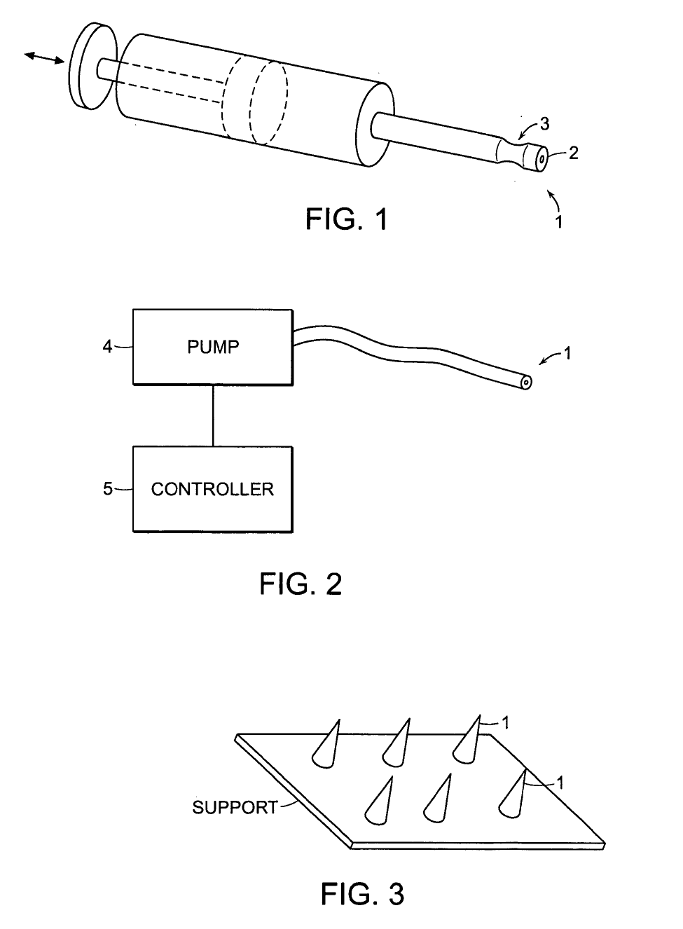

Accordingly, in some embodiments, a cell introduction device may be based on a typical syringe or printer device that is modified to provide one or more additional structural and/or functional features adapted for cellular delivery. Alternatively, in some embodiments, a cell introduction device is an integrated device that does not resemble a typical syringe, but incorporates one or more features that are designed to protect cellular function and/or assist in the delivery (e.g., from a syringe or printer) of functional cells to an organ or tissue site. In some embodiments, a device is provided that comprises one or more microfluidic channels or circuits. In some embodiments, a device (e.g., a device comprising one or more microfluidic channels or circuits) is provided that is designed and configured for preparing, defrosting, and/or injecting cells. In some embodiments, a cell introduction device comprises one or more working ends that can deliver cells to a target tissue site. The working ends include one or more openings that are sufficiently large to allow cells to be delivered to the tissue site. The working ends can be connected via a fluid pathway to a component (e.g., a pump, a syringe plunger, or other actuator) that can cause fluid to flow through the fluid pathway and out of the opening. In some embodiments, a cell introduction device may be configured to monitor cells being delivered, to regulate the environment of the cells (e.g., their temperature, oxygen level, toxin level, nutritional level, fluid composition, pH, etc., or any combination thereof), to provide feedback and/or control related to the injection process (e.g., pressure, time, volume, etc., or any combination thereof), and/or to evaluate the tissue target site. In some embodiments, one or more of these functions may be provided by components (e.g., temperature regulators, pumps, controllers, filters, sensors, power supplies, etc., or any combination thereof) that are integrated into the cell introduction device. In some embodiments, one or more of these functions may be provided by separate components that are configured with the cell introduction device to provide a cell introduction system that performs one or more of the functions described herein to assist in the delivery of functional cells to a target site. It should be appreciated that the different configurations of a cell introduction device described herein may be combined with one or more additional components as described herein. It also should be appreciated that particular structural or functional features described in the context of one embodiment may be used in combination with an alternative embodiment, unless otherwise indicated or unless the embodiments are incompatible. FIGS. 1-3 illustrate non-limiting embodiments of different configurations of cell introduction devices that may be used or adapted as described herein. However, alternative configurations may be used as described herein.

In some embodiments, fluidic devices can store and prepare cells for freezing, defrosting, reconstitution, and/or clean-up for injections. In some embodiments, fluidic devices can be used for injecting cells into or onto target.

Configurations of the Working End of a Cell Introduction Device:

In some embodiments, the working end 1 of the cell introduction device includes a tube with an opening 2 at a distal end as shown in FIG. 1 and described in more detail herein. The tube may be flexible or rigid. In some embodiments, a cell introduction device includes a working end 1 that is fluidly connected to a component 4 such as a pump (or other device that can substitute for the pump, and/or other components such as a controller, power supply, etc., or any combination thereof) as illustrated in FIG. 2 and described in more detail herein. The working end 1 may be connected to component 4 in any suitable way. In some embodiments, this allows the working end to be placed at the site of delivery on a moving tissue or organ (e.g., pulsating heart) while the pump and/or one or more other components (that may be integrated into a single apparatus, or combined to form a system) may be placed on a stable surface and remain connected to the working end via a flexible member. In some embodiments, a cell introduction device may include a plurality of working ends, each with at least one opening as illustrated in FIG. 3 and described in more detail herein. A plurality of working ends (e.g., as illustrated in FIG. 3) can be incorporated into a device such as the one illustrated in FIG. 1 (e.g., in an embodiment having a rigid tube with a plurality of working ends) or at the end of a flexible member such as the one illustrated in FIG. 2.

FIG. 3 shows an illustrative embodiment of a cell introduction device that includes a plurality of working ends each with at least one opening. In this illustrative embodiment, the plurality of working ends each have the form of a tapered needle-like-like structure extending from a support, but of course may have a straight or non-tapered configuration, gimlet arrangement or other, as desired. In some embodiments, the cell introduction device comprises an injector in a patch format with one or more injection orifices. In some embodiments, the cell introduction device comprises an injector having a single hole patch. In some embodiments, the cell introduction device comprises an injector having a fixed needle like structure. In some embodiments, the cell introduction device comprises a combination of a fixed needle like structure and a patch comprising one or more injection orifices.

Also, although the working ends are shown arranged at approximately a 90 degree angle to the support the working ends may be arranged at any suitable angle or angles, e.g., an angle that provides suitable penetration into tissue and helps to prevent leakage of cells from the tissue site. In some embodiments, the working angle is optimized for the location of the injection site. In some embodiments, the working angle is optimized for injection into a particular organ. Each of the working ends may have a channel or other passageway along which cell fluid may be moved and introduced at a tissue site. Although in this embodiment the working ends are arranged in a rectangular array, other arrangements are possible, such as a linear array, a circular array (e.g., to permit introduction of cells around a circular periphery of a damaged tissue site), and others. The array of working ends may be constructed and arranged to be secured to a tissue with the working ends extending into the tissue during release of cells. For example, the array of working ends together with the support may be secured to a heart tissue with the working ends extending into the tissue. Thereafter, with the working ends and support fixed to the (potentially moving) heart tissue, cells may be introduced at the tissue site via the working ends. In this embodiment, the working ends may receive cell fluid from a pump that is remote from the working ends, or that is mounted to the support. In some embodiments, the pump may be an electric pump, an electro-osmotic pump, or an osmotic pump. However, other types of pumps may be used.

It should be appreciated that regardless of the configuration used, each of the one or more working ends may have a rigid elongated member (e.g., a needle) at its tip that has an opening with an appropriate diameter for delivering cells. In some embodiments, the tip of the working end may be blunt. In some embodiments, the tip of the working end may be needle-like, e.g., tapered, pointed, or sharp to help penetrate the tissue at the site of injection. The length and diameter of the tip may be different depending on the application, as described in more detail herein.

In some embodiments, any configuration of one or more working ends may be arranged to include one or more features or in combination with one or more additional components to provide further functionalities as described herein.

### Filtration:

In some embodiments, the opening(s) at the working end of a device are open. However, in some embodiments, one or more filtration layers are deployed at the opening(s) to allow cells (e.g., cells of a desired size or shape) to pass through while retaining unwanted material (toxins, cellular debris, waste products) etc., or any combination thereof. In some embodiments, material that closes or occludes the opening(s) may be a material having a membrane-like structure (e.g., for filtration or dialysis), or one or more layers of beads, gels, chemical additives, or other features that could trap or release unwanted contaminants or debris or chemicals that can or should be released, for example, while still allowing cells to pass through. In some embodiments, a membrane may be treated with chemical compounds. In some embodiments, the membrane is not treated. In some embodiments, membranes (treated or non-treated) are selected to i) absorb chemical cues in the cellular solution that come from dying cells, ii) or absorb toxins from the cellular solution, and/or iii) filter and prevent debris from entering the target tissue. The membranes may be size-exclusion membranes in some embodiments. However, in some embodiments a membrane structure may enclose a filtering configuration that removes smaller debris and allows cells to pass (e.g., a bed of beads having small pores that allow the debris to penetrate but do not allow the cells to penetrate). In some embodiments, a size-exclusion packing may be used to create a tortuous path that results in the separation of cells from contaminants without creating an undesirable back pressure. It should be appreciated that debris may include toxic waste and/or biological compounds secreted by cells (e.g., growth and/or regulatory factors) and/or ions or other molecules.

It should be appreciated that one or more membranes or other filtration configurations may be attached to the working end of a delivery device using any suitable method or technique, including, but not limited to, glue or other adhesive, one or more mechanical fasteners, physical barriers (e.g., one or more layers of porous plastic, glass, or other material) that can capture and retail a filtration medium while still allowing cells to pass through. It should be appreciated that in some embodiments, the working end of a cell delivery device may be manufactured to contain an integrated barrier (e.g., a porous barrier) that can serve to retain a filtration medium. In some embodiments, one or more rings, ridges, grooves, protrusions, or other structures on the internal wall of the working end may be used to retain a pre-packed cartridge that can act as a filter (e.g., it contains appropriate filtration material within a membrane or other porous support). It should be appreciated that any appropriate size exclusion may be achieved. In some embodiments, a filtration medium is provided to capture material smaller than about 0.25 microns in diameter in order to capture cellular debris but let the cells go through. In some embodiments, a filtration medium is provided to capture smaller peptides (e.g., growth inducing peptides or other peptides, for example using an approximately 3,000 Da molecular weight cutoff) while letting cells go through.

In some embodiments, one or more of these features are used in the cell preparation stage to treat and/or filter a sample as it is brought into the injection device. In some embodiments, one or more of these features is included in the body of the cell introduction device (e.g., in the reservoir, in a channel leading to the working end, at the tip of the working end, in any other suitable location within the device, or any combination of two or more thereof). In some embodiments, one or more of these features is used i) to process a cellular preparation prior to loading it into a cell introduction device, ii) to process the cells as they are being introduced at a tissue site from the working end of the device, or a combination of i) and ii). One or more membrane and/or filtering structures may be present in each working end of a cellular introduction device (e.g., whether the device has a single working end or an array of working ends). However, it should be appreciated that a device may have one or more such filters at other locations to keep the cells in the cellular solution as healthy as possible and remove any material that may interfere with a successful cellular delivery.

Configurations for Protecting Cells and/or Tissue Sites from Damage:

It should be appreciated that in some embodiments of the invention the diameters of certain working ends and/or other tubular structures of the invention are sufficient to allow cells to pass through, e.g., without undue shear stress. In some embodiments, the internal diameter of a needle-like member or other tubular structure may be at least 5 microns, about 5-10 microns, 10-25 microns, 25-50 microns, 50-100 microns, or larger.

In some embodiments, the working end is as short as possible to minimize physical stress or shearing during administration. In some embodiments, the length of the working end or other tubular structure is designed to be sufficient to deliver cells to a target region, but not significantly longer. This reduces the distance that the cells travel through the confines of the working ends or other tubular structure, thereby avoiding excessive shear stress. For example, a needle-like member or other tubular structure may be between 1 mm and 5 mm (e.g., 1, 2, 3, 4, or 5 mm) long as described herein. A typical working end currently has a length that greatly exceeds its diameter (especially its internal diameter) by a factor of 10× or more. In contrast, a needle-like member or other tubular structure used in connection with any of the devices and embodiments described herein may be only approximately 1 millimeter long. The minimum length of the working end is the maximum depth of tissue penetration for a particular application. In some embodiments, heart injections involve a depth of tissue penetration on the order of 1-2 mm. However, the length may be shorter in some embodiments. For example, length on the order of a fraction of a mm may be used for certain applications where injection into a thin tissue layer is required (e.g., injection into a myelin layer to promote myelin regeneration surrounding a nerve).

However, it should be appreciated that longer working ends or tubular structures may be useful for certain applications. For example, in some embodiments the size of the needle may be selected to allow the injection to proceed along the path of least destruction in the receiving tissue. This may be particularly important, in some embodiments, for injections into the brain where tissue damage should be minimized. Accordingly, long working ends (e.g., on the order of several inches, e.g., up to about 8 to 10 inches long or up to about 20 to 25 cm long may be used for certain applications). Also, in some embodiments having multiple needle configurations, a sliding distance or injection stop may be used to re-enforce the long physical needle structures.

In some embodiments, the shape of the syringe and working end are designed to minimize zones of stress or shear that could damage the cells during injection. In some embodiments, an injector is designed to avoid significant or irregular pressure gradients within the injector. For example, the internal volume of an injector may be designed to avoid or reduce sharp transitions of the internal diameter. In some embodiments, the internal volume of an injector may be regularly tapered from the reservoir end to the opening at the distal end. In some embodiments, an injector is designed to avoid internal features such as edges, sharp angles, or protrusions that produce shear stress on cells within the injector.

In some embodiments, an injector or a portion thereof includes features that promote a regular pressure gradient. For example, the diameter of the opening at the distal end may be as wide as possible to reduce shear when the cells are introduced at the target site. These and other features are described in more detail herein.

In some embodiments, the material of the injector or a portion of the injector (e.g., the working end, the reservoir, or both) is selected to minimize interactions with the cells thereby to avoid unnecessary shear stress due to cells sticking to the inside of the injector. In some embodiments, the material is inert. In some embodiments, the inner surface of an injector or a portion of an injector is coated with an agent or material that is selected to avoid or reduce interactions with a cell (e.g., PTFE coating and/or heparin).

In some embodiments, the injection working end is designed to minimize both physical tissue trauma and biochemical or physiological trauma at the site of injection. For example, an injector may be designed to minimize the recipient's response to trauma associated with the injection (e.g., the recruitment of neutrophils, white blood cells, cytokines and other inflammatory or healing responses that might reduce the survival of the injected cells). In some embodiments, a needle-like member or other tubular structure that is used to deliver cells is designed to be small (e.g., narrow and/or short) to minimize tissue damage at the target site in the recipient. In some embodiments, the needle-like member or other tubular structure has an internal diameter that is approximately the size of the cells being injected. In some embodiments, the diameter is only somewhat larger than the diameter of the cells being delivered (e.g., 2-5 times the cell diameter) to avoid undesirable physical stress on the cells while also minimizing damage to the recipient tissue). It should be appreciated that different cells have different average dimensions. For example, a typical stem cell is 5-10 microns in diameter, whereas other cells may be larger (e.g., an oocyte may be on the order of 150 microns in diameter). Accordingly, different internal diameters may be used for different cells.

In some embodiments, the material of the injector or a portion thereof (e.g., the needle-like member or other tubular structure) is selected so that the outside diameter can be as small as possible but still provide sufficient structural integrity. In some embodiments, working end has an internal diameter of 20-100 and an external diameter of 50-150 microns (e.g., approximately 36 gauge or higher).

In some embodiments, an injector is designed to reduce or avoid dead space volume. In some embodiments, the needle-like member or other tubular structure contains the entire injection volume. In effect, the working end and syringe are not separate but rather the working end is the syringe. Accordingly, in some embodiments, the injector consists of a long thin hollow tube connected to a plunger, a pump, or other fluid displacing device.

In some embodiments, a needle-like member or other tubular structure is designed to prevent coring of the flesh and/or is designed to minimize trauma to the tissue at the site of injection, thereby minimizing the host physiological response. In some embodiments, the physical shape of the needle-like member or other tubular structure is designed to minimize trauma. In some embodiments, the surface of the needle-like member or other tubular structure is designed to minimize trauma (e.g., it is smooth). In some embodiments, the material or surface coating of the needle-like member or other tubular structure is designed to reduce adhesion to tissue at the site of administration (e.g., the material may be coated with PTFE or other non-adhesive material). In some embodiments, the material or surface coating may be non-immunogenic.

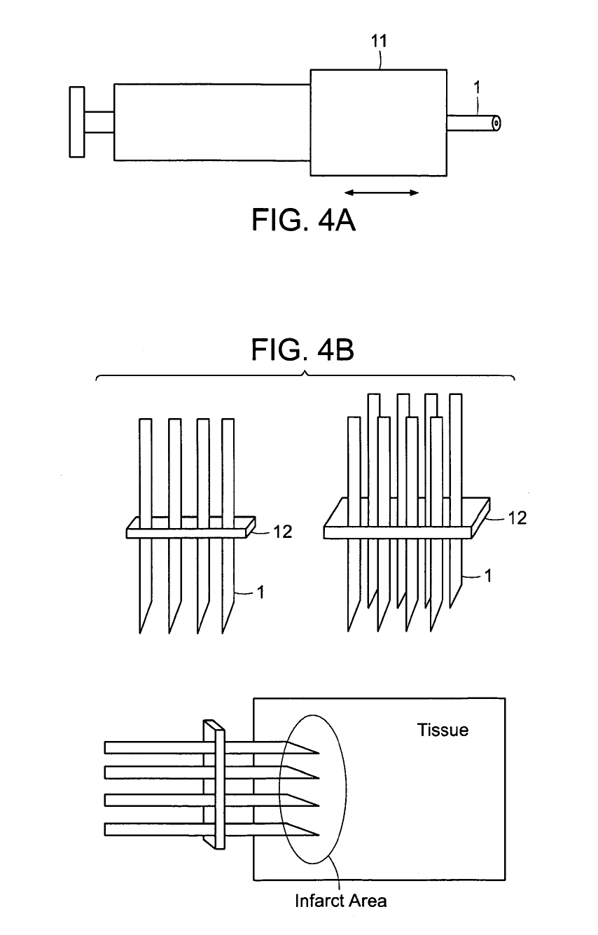

In another aspect of the invention, a working end of a cell introduction device may be associated with a movable stop or other component that limits a depth to which the working end may be inserted into a tissue. For example, as shown in FIG. 4A, a syringe-type device may include a movable sleeve 11 that may be positioned relative to the working end 1 so that the sleeve 11 contacts the tissue surface when the working end 1 has penetrated the tissue to a desired depth. The sleeve 11 may be mounted to the syringe body and fixed at multiple different positions to provide different working end depths. In some embodiments, a sleeve 11, or similar structure (e.g., a stop), may be used in either a multi-needle or single needle configuration as a support for needles, particularly relatively long needles, to prevent bending of the needles. For example, the sleeve 11 may be threadedly mounted to the syringe body, allowing rotation of the sleeve 11 to move the sleeve 11 axially relative to the working end 1. Alternately, the sleeve 11 may engage the body with an interference fit such that friction maintains the sleeve 11 in place, but allows a user to move the sleeve if desired.

FIG. 4B illustrates a non-limiting embodiment of an array of working ends (e.g., needles) that is held in place with a support member 12 comprising a plurality of openings through which the working ends are inserted. In some embodiments, this support member 12 provides structural support to maintain the structural integrity of the working ends and avoid bending or distortion of one or more working ends that could interfere with the effectiveness of the device. In some embodiments, the support member 12 also may provide a “stop” that prevents the working ends from being inserted into underlying tissue beyond the location of the support member 12 along the axis of the working end. In some embodiments, the support member 12 may be at a fixed position. In some embodiments, the support member 12 may be adjustable and movable along the length of the working end to provide for different depths of injection depending on the application. A support member 12 may be used in association with any array configuration of multiple working ends (e.g., needles). It may be useful to provide structural integrity and a maximum depth of penetration for use with any tissue (e.g., heart, brain, skin, etc.). It may be used for injection in a localized linear space or plane or any other configuration with multiple ends. The working ends can be metal, carbon, plastic, etc., or any combination thereof. The working ends can be arranged in any array configuration.

Configurations with a Working End Connected Via a Flexible Member to One or More Additional Components (e.g., Pumps, Controllers, Detectors, or Other Components):

In some embodiments, the working end of a device may be connected to other components (e.g., pumps, controllers, etc., or any combination thereof) via a flexible member (e.g., tube).

FIG. 2 shows a schematic diagram of an embodiment of a cell introduction device that incorporates one or more aspects of the invention. In this illustrative embodiment, the cell introduction device includes a working end 1 that is fluidly connected to a pump 4 (or other device that can substitute for the pump). The working end 1 may be connected to the pump 4 in any suitable way, such as by a rigid tube, channel or other conduit, by a flexible tube, by a multi-channel manifold, or other capable of transmitting fluid pressure from the pump to the working end. The pump 4 may also be arranged in any suitable way, and may include one or more peristaltic pumps, syringe pumps, osmotic pumps, and/or any other arrangement to cause flow of cells at the working end 1. In one embodiment, the pump 4 may move air or other fluid at the working end such that the pump 4 may aspirate or draw cells into the working end from an external source. Thereafter, the pump 4 may move air or other fluid in an opposite direction to dispense cells at the working end. Thus, cell fluid need not necessarily contact the pump 4, which may aid in maintaining a suitable environment for the cells. Alternately, cell fluid may be provided directly from the pump 4 to the working end 1, e.g., a reservoir of cell fluid may feed the pump 4, which moves the cell fluid from the pump 4 to the working end 1. The plumbing at the pump 4 and working end 1 may include various manifolds and other arrangements to allow the pump 4 to introduce different fluids, such as a priming fluid that may be introduced at the tissue site prior to cells being placed, or fluids added to the tissue site after cell placement, e.g., to feed or oxygenate the cells, provide growth factors, removed toxins, and so on. Thus, a manifold and valving arrangement may permit the pump 4 to provide different fluids to the working end. In some embodiments, remote needle ports can have vacuum cup or a vacuum tube to assist in holding the device onto organs and/or tissues for extended periods.

### Configurations for Positioning a Working End at a Tissue Site:

In some embodiments, aspects of the invention relate to techniques for positioning a working end (e.g., with a single opening or an array of openings) at a tissue site for improving the delivery of a cellular preparation.

In some embodiments, a robotic system may be used to position a working end at a tissue site. In a non-limiting embodiment shown in FIG. 2, the controller 5 may include a robotic system or other arrangement to position the working end 1 at a desired location at a tissue site. For example, in a case where the controller 5 inserts the working end 1 into a portion of a live, beating heart (or alternately breathing lungs or other moving tissue), the controller 5 may need to compensate for movement of the heart tissue during and/or after deployment of the working end 1 at the tissue site. To do so, the controller 5 may include a robotic vision system, infrared sensor, or other arrangement that detects movement of the tissue site of the heart where cells are to be introduced and controls movement of the working end 1 so that the working end 1 is inserted into the heart tissue at the proper location and/or so that the working end 1 moves with the tissue location as the heart beats or otherwise moves appropriately. That is, in many cases it may be important that cells are introduced not only at the correct surface location of the heart, but also at the appropriate depth in the heart tissue. Since the desired tissue site may move, once an appropriate tissue site is identified, the controller 5 may track the position of the tissue site, even as it moves, and move the working end so that it is properly placed at the tissue site, and remains in the proper position during cell introduction. The controller 5 may also assist in ensuring that the working end 1 is inserted into the tissue at an appropriate angle, since in some applications the working end 1 should be arranged at a particular angle to the tissue surface. For example, in the case of a heart tissue, the working end should be inserted into the heart tissue at a relatively low angle and to a specific depth below the tissue surface. Where the surgeon manually manipulates the cell introduction device, the surgeon may use tactile feedback to determine when the working end is at an appropriate location, such as resistance of the heart tissue to the inserted working end, rate of travel of the working end in tissue, a resistance of the working end to rotation once placed in the tissue, etc. The controller 5 may include sensors, displays, actuators or other devices to provide the surgeon with feedback, and/or may insert the working end 1 into the tissue in a fully or partially automated way. For example, the controller 5 may detect a force of the heart tissue on the working end (indicating resistance of the tissue to insertion of the working end) and limit movement of the working end so that there is an upper limit to the level of force used to insert the working end. Thus, the surgeon may focus only on the angular position of the working end and rate of travel of the working end when inserting into tissue, and rely on the controller 5 to ensure that insertion force limits are not exceeded. In this example, the controller 5 may provide a visual and/or audible indication when an insertion force is reached, or may actually prevent the application of excessive force, e.g., by retracting the working end, by resisting movement of the surgeon's hand, etc. Of course, this is only one example. The controller 5 may be used to control other aspects of cell introduction, such as the angle at which the working end is introduced into the tissue, a range of motion of the working end, and so on.

In another embodiment, the cell introduction device may compensate for tissue site movement by mounting the working end to the tissue site, and having a flexible connection (e.g., a tube made of rubber, a polymer, etc., or any combination thereof) between the working end and the pump. Thus, the working end 1 may move with the tissue site, and the flexible connection to the pump may allow not only application of pressure or other force to move cells at the working end 1, but also permits the working end to move with the tissue site without interference by the pump or other portions of the cell introduction device. In yet another embodiment, the pump and working end may be fixed together and mounted at the tissue site so that the pump and working end may move together as the tissue site moves. For example, the pump 4 and working end 1 may be fashioned into a sort of patch that is applied to the tissue and fixed in place, e.g., by an adhesive, suture, vacuum/suction, an elastic band, grease or other mechanical fastener. The controller 5 may be remote from the pump 4 and working end 1, and may communicate with the pump 5 by wired and/or wireless communication. Alternately, the controller 5, or at least a portion of it may be fixed together with the pump and working end.

In some embodiments, a hand-held or stereotaxic mounting may be used.

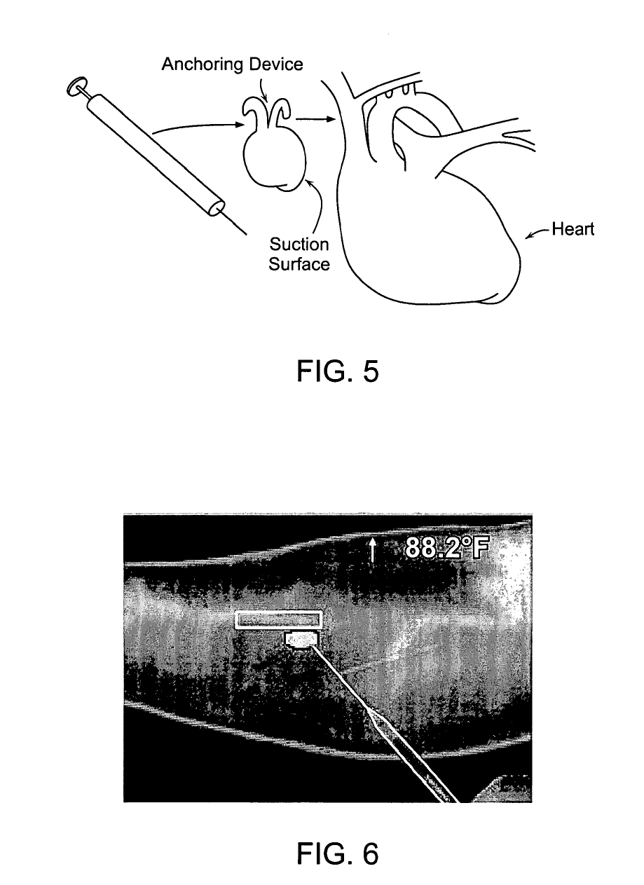

In some embodiments, the cell introduction device may include a mounting device that secures the cell introduction device to a tissue or other body structure so that the working end may be suitably positioned relative to the tissue. For example, as shown in FIG. 5, a bracket or anchoring device may be arranged to engage with the superior vena cava, pulmonary artery, aorta or other body structure so as to support a syringe-type or other cell introduction device on a heart with the working end of the cell introduction device inserted into a portion of the heart. Since the bracket or anchor may support the cell introduction device on the heart, the device may move with the heart (or at least the working end may move with the heart), allowing the working end to remain in a desired location and at a desired depth in the tissue. In one embodiment, the anchor may include a suction surface that engages with the heart or other tissue with a suction force that maintains the anchor and cell introduction device in contact with the heart. For example, a vacuum may be applied to the anchoring device suitable to secure the anchoring device in place without detrimentally affecting heart function. It should be appreciated that other structures may be used to attach a device (e.g., the working end of a device) to a heart or other organ.

In some configurations, an injector (e.g., a syringe and needle-like member, or any other suitable injector) may be combined with a micro-positioning device (e.g., a mechanized micro-positioning device). The working end can be advanced into the recipient tissue or flesh by a motorized positioning system, and stopped at the injection site. In some embodiments, when the injection commences, the fluid is injected at the same time as the micro-positioning system withdraws the working end from the tissue or flesh. The rate of liquid infusion can be matched to the rate of working end withdrawal so as to put very little pressure on the cells (e.g., no more pressure than that of the surrounding tissue or flesh). In certain embodiments, the process may be matched so as to locate the cells along the line of the working ends path or some fraction thereof. Accordingly, cells could be injected into a cavity created by the working end (e.g., a cavity that is approximately 50 microliters in volume even if the entire cavity is much larger, e.g., 100 micoliters). In some embodiments, a tissue site may be prepared for injection by removing a small volume of cells. Certain devices may include a port for coring out a column of tissue or cells as injection occurs. In other embodiments, a core of cells may be removed prior to injection and the injector tip is introduced at the site of cell removal. It should be appreciated that the use of a micro-positioning device, particularly a mechanized device, can be helpful in this procedure, but is not required.

Using Temperature to Track Injections and Additional Material:

Aspects of the invention provide methods and devices for tracking the injection path of agents, e.g., drugs or cells, in an organism. In some embodiments, the injection path of an agent is tracked by evaluating differences in temperature between an injected fluid and a surrounding tissue. In some embodiments, the actual injection path of a molecule (e.g., protein, nucleic acid, small molecule, drug) or cell (e.g., stem cell) solution is tracked in an organism, organ, or tissue. In some embodiments, devices and methods are provided that detect relatively small temperature differences between an injected fluid and an ambient or surrounding environment, e.g., tissue environment. In some embodiments, devices and methods are provided that detect temperature differences between an injected fluid and a surrounding environment of at least 0.000001° C., at least 0.00001° C., at least 0.0001° C., at least 0.001° C., at least 0.01° C., at least 0.1° C., at least 1° C., or at least 10° C. In some embodiments, devices and methods are provided that detect temperature differences between an injected fluid and a surrounding environment in a range of 0.00001° C. to 0.0001° C., 0.00001° C. to 0.001° C., 0.00001° C. to 0.01° C., 0.00001° C. to 0.1° C., 0.00001° C. to 1° C., 0.0001° C. to 10° C., or 0.001° C. to 100° C. Accordingly, the solution being delivered may be prepared to be from about 0.00001 to about 10 C. higher or lower than the expected temperature of the tissue site (e.g., from about 0.00001 to about 0.0001, from about 0.0001 to about 0.001, from about 0.001 to about 0.01, from about 0.01 to about 0.1, from about 0.1 to about 1.0, from about 1.0 to about 5.0 C, higher or lower).

Infrared imaging technology may be used, for example, to detect and optionally image such differences. Any of the infrared devices disclosed herein may be used, for example. Thus, in some embodiments, a temperature difference between an injected fluid and a surrounding environment is displayed in an infrared image. In some embodiments, the image depicts a temperature or wavelength map. In some embodiments, the image depicts penetration and/or distribution of an injected fluid in an surrounding environment using a cartesian coordinate system (e.g., x, y and z co-ordinates, x and y coordinates, x and z coordinates, y and z coordinates). FIG. 6 depicts an illustrative map showing injection flow path in which the intensities correspond to temperatures.

In some embodiments, devices and methods are provided for evaluating an injection site. In some embodiments, devices and methods are provided to visualize flow paths of an injected fluid around or near an injection site of a tissue. For example, a solution that is injected into a tissue may be imaged based on differences in temperature between the solution and surrounding tissue environment. In some case, the dynamics of temperature change within a tissue following injection of a solution into the tissue may be evaluated. Infrared imaging technology may be used, for example, to detect and optionally image such temperature differences. Any of the infrared devices disclosed herein may be used, for example.

The flow path of an injected solution in a tissue and/or the dynamics of temperature change in the vicinity of the injection site may provide information regarding the quality and/or status of the tissue. For example, the flow path of an injected solution in a tissue and/or the dynamics of temperature change in the vicinity of the injection site may provide information regarding the structure, porosity, permeability, vascularity, metabolic activity, etc. of the tissue. In some embodiments, information regarding the quality and/or stage of the tissue serves as an input for a control system that controls injection into the tissue. In some embodiment, the information is used to optimize an injection protocol. In some embodiments, the information is used to determine the viability of injected agents (e.g., cells) at the injection site.

In some embodiments, a relatively cold fluid is contacted with the surface of the tissue. The surface temperature of the tissue is monitored over time before, during and/or after contacting the surface of the tissue with the relatively cold fluid. During this time, the surface temperature of the tissue changes. In some embodiments, the dynamics of this temperature change provides insight into the structure, health and/or content of the underlying tissue. In some embodiments, higher temperature areas of a tissue return to temperature faster than lower temperature areas. Thus, in some embodiments, a comparison of images obtained over time can identifying relatively hot and relatively cold areas of a tissue. In some embodiments, the relatively high temperature areas correspond to relatively highly vascularized regions and/or relatively high metabolic activity.

In certain embodiments, devices and methods are provided to assess the quality and/or status of a tissue at or near an injection site based on spectral energies. Spectral energies may be measured, in some embodiments, to evaluate the distribution of different molecules (e.g., O₂, Hemoglobin, myoglobin, glucose) within a tissue and/or near an injection site. Infrared imaging technology may be used, for example, to detect and optionally image such spectral energies. Any of the infrared devices disclosed herein may be used, for example.

Similarly, relatively higher temperatures may be used in some embodiments.

It should be appreciated that in some embodiments cells are injected into fringe areas surrounding dead tissue (e.g., fringe areas surrounding dead or dying cells in an infarcted heart), because the viability of injected cells may be severely reduced if they are injected directly into dead or dying tissue.

In some embodiments, injections are made at a shallow angle into the tissue (as opposed to injecting at a right angle relative to the plane of the tissue) in order to increase the probability of injecting into surface layers that are targeted. Accordingly, in some embodiments, the working end(s) of a device may be at a shallow angle relative to a support structure (e.g., relative to the plane of the surface of an array to which the working ends are attached). It should be appreciated that in some embodiments, the angle formed between the plane of a first surface of a support structure and the axis of each working end may be the same (e.g., about 10, about 20, about 30, about 40, about 50, about 60, about 70, about 80, or about 90 degrees). In some embodiments, all of the working ends have the same orientation. However, in some embodiments, different subsets of working ends on an array may be at different angles relative to the plane of a surface of the support structure and/or may be oriented in different directions. In addition, the lengths and/or cross-sectional areas of the different working ends may be different. Such non-uniform arrays may be useful to provide injection depths and configurations that are adapted to particular tissue applications (e.g., due to the geometry of the tissue). It should be appreciated that the support structure may be flexible or rigid. In some embodiments, a rigid support structure may be shaped to conform to the shape of a tissue to which it will be applied.

In some embodiments, working ends that form an angle of less than 90 degrees (e.g., from 10-80, around 20, around 30, around 40, around 50, around 60, or around 70 degrees) relative to the surface of the support may be inserted into the underlying tissue by moving the array sideways along the surface of the tissue after making contact with the tissue. Appropriate pressure for this application could be determined by one of ordinary skill in the art.

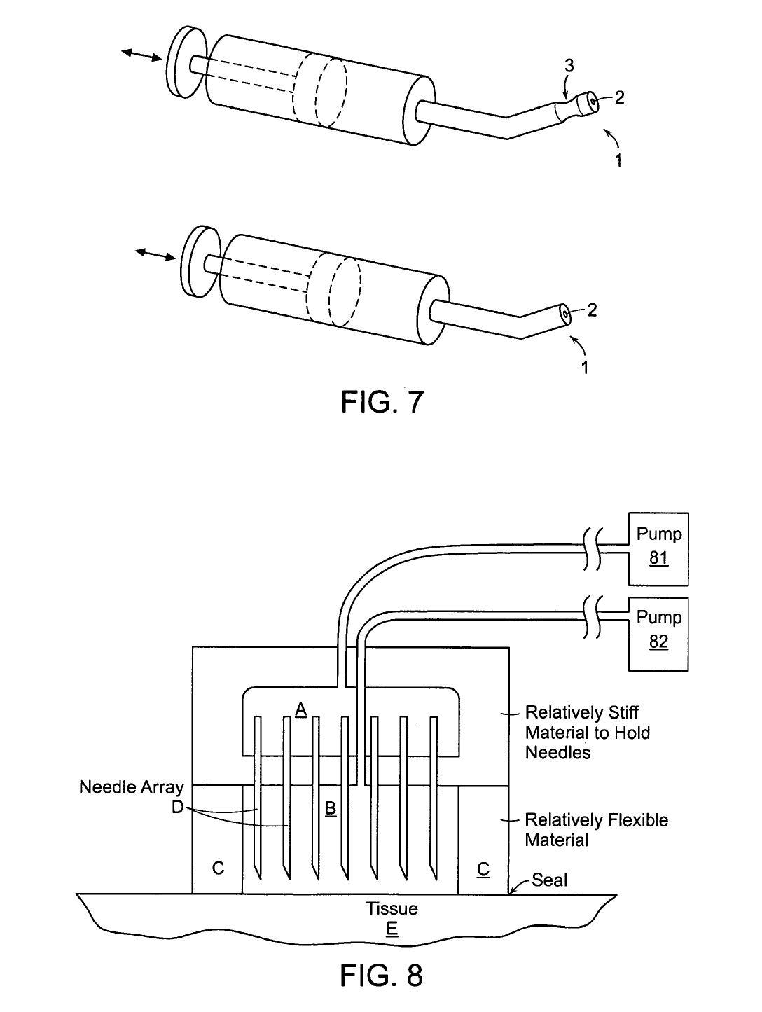

In some embodiments, the angle of the working end(s) relative to the support is achieved by using one or more needles that are bent or curved to create an appropriate angle between the tip at the distal end of the working end that contacts the tissue and the support that is attached to the proximal end of the working end. FIG. 7 illustrates non-limiting examples of bent or curved needles. However, it should be appreciated that any suitable angle may be implemented (e.g., between 90 and 180 degrees, for example between 100 and 170, about 110, about 120, about 130, about 140, about 150, or about 160 degrees).

### Configurations for Maintaining a Working End at a Tissue Site:

In some embodiments, a cell introduction device has a syringe-type configuration that is shaped to protect cells at the site of injection. In accordance with an aspect of the invention, the working end may have a feature that helps to maintain the working end in place at a tissue site, that helps to prevent leakage or other unwanted movement of cells at the tissue site, and/or that helps to reduce an introduction pressure required to place cells at the tissue site. For example, in some embodiments, the tube may have a recess 3 in a region adjacent to and proximal of the distal end. The recess 3 could be arranged in a variety of ways, such as a circumferential groove or grooves, a longitudinal groove or grooves, a conically-shaped portion of the tube, and others. The recess 3 may provide a pocket in the tissue for fluid exiting the opening 2 to initially collect, allowing the fluid to exit from the opening 2 at a lower pressure than would otherwise be required. That is, when the working end is initially introduced at the tissue site, tissue may be pressed against the opening 2 and other portions of the working end, resisting the movement of cells from the opening 2 and into the tissue. The recess 3 may provide a void into which cells may at least initially move, thus reducing the pressure that might otherwise be needed to move cells from the opening 2. Alternately, or in addition, the recess 3 may provide an improved seal between the working end 1 and the surrounding tissue, potentially helping to prevent fluid from exiting or blowing back up the injection path from the tissue site along an interface between the working end and the tissue. For example, the recess 3 may be formed as a reduced diameter section of the tube that allows the tissue to bulge into the recess 3 and form a seal between the tissue and the working end 1. As a result, cells introduced at the tissue site under pressure may be contained at the tissue site and prevented from traveling along a space between the working end and the tissue. In some embodiments, the device is designed and configured to absorb and/or dissipate pressure to prevent blow-back (e.g., by producing a pressure ridge that acts like a compression o-ring). It should be appreciated that other configurations of ridges, grooves, shapes, protrusions, or any combination thereof may be included at the working end (e.g., on a needle) in order to prevent fluid flowing back up the sides of the working end after delivery (e.g., up the side of a needle after injection). These may be designed such that the tissue being penetrated can conform to create pressure ridges (e.g., so that the injected fluid would have to be forced by thereby preventing leaks).

In some embodiments, an array may be designed to adhere to an organ or tissue surface (e.g., a surface of the heart) so that it moves with the tissue or organ (e.g., it moves with the heart as it beats) thereby removing the need for moving the current needle/syringe in time with the heart beats which can be challenging. In some embodiments, adherence (e.g., to the heart) may be accomplished using an adhesive material (e.g., a glue—for example, a lightly sticky glue like could provide sufficient adherence, but be releasable, for example by pumping a release solution down the line and between the patch and the tissue). In some embodiments, adherence may be accomplished by drawing a slight vacuum into a space that contains the array. FIG. 8 illustrates a non-limiting embodiment of an injector array with a vacuum for attaching to a tissue. In some embodiments, application of a vacuum could be used to drive the needles (in a controlled fashion) into the tissue to a known depth (depending on the strength of the vacuum and the compressibility of the plastic wall material shown in FIG. 8. The penetration depth may be limited by the geometry of the device and the compressibility of the materials. The device could be released at the end of the injection by releasing the vacuum. The entire device could then be retrieved by pulling on the fluid lines. In more detail, with reference to FIG. 8, compartment A may be filled with a drug or cell suspension. Compartment B may initially be filled with air but upon application of slight vacuum from pump 82) the device is attached to the tissue surface. Upon further application of vacuum, wall C compresses delivering needle array D controllably for a known and limited distance into tissue G. Pump 81 then delivers cells etc. into the tissue. The device can be removed by releasing the vacuum. Other configurations of this embodiment also may be used.

### Configurations for Maintaining Appropriate Pressure Profiles During Injection:

In one aspect of the invention, regardless of the cell injector system that is being used, control over introduction of cells at a tissue site may be adjusted to help enhance the survival of the cells, the likelihood that the cells will remain in a desired location or other characteristics. For example, in some applications, the cells may be delivered to the tissue site at a constant pressure, or at a pressure below a threshold level, at a constant flow rate, or at a flow rate below a threshold level, over a delivery time. Thus, the pump may be controlled to maintain a constant pressure and/or flow rate of the cell fluid at the working end during cell introduction at the tissue site. As described herein, the pressure of the cell fluid may vary during introduction of cells, e.g., because of movement of tissue, leakage or other movement of cell fluid into voids in the tissue, and so on. Using feedback control of the pump, the cell fluid pressure and/or flow rate may be adjusted as needed. The pump may be operated to apply positive pressure to increase pressure at the working end, or to apply negative pressure to reduce pressure at the working end if necessary to maintain pressure at a constant level (or maintain pressure below a threshold level).

In some embodiments, the pressure during injection is limited below a maximal pressure threshold that is physiologically relevant. For example, a pressure threshold may be set to maintain the pressure of the injected material at no higher than blood pressure. In some embodiments, the pressure threshold may be selected as an average blood pressure. In some embodiments, the pressure threshold may be set at the high end of the range of physiological blood pressures. In some embodiments, a threshold may be patient specific and selected to correspond to the blood pressure of the patient. In some embodiments, the blood pressure of the patient may be monitored during the injection process and the pressure threshold may be adjusted during the injection process. In some embodiments, the pressure threshold may be set as a function of the type of cells that are being injected. According to aspects of the invention, different cell types may have varied sensitivities to pressure. In some embodiments, the pressure threshold may be set as a function of the tissue site at which the cells are being introduced. In some embodiments, a pressure threshold may be set at between 100 mm mercury and 200 mm mercury (e.g., about 100, 110, 120, 130, 140, 150, 160, 170, 180, 190, or 200 mm mercury). However, higher or lower pressure thresholds may be used as the invention is not limited in this respect. Unless otherwise indicted, or apparent from context, pressures disclosed herein are gauge pressures.

In some embodiments, a pressure threshold may be set using a feedback system. For example, a controller may receive input from a sensor in the injector (e.g., in the working end) and regulates the amount of pressure imposed on the cells being injected (e.g., via a pump, plunger, or other actuator). In some embodiments, a pressure threshold may be set using a physical valve or other component that prevents pressure above the threshold level from being exerted on the cells in the injector (e.g., in the reservoir and/or syringe working end). However, other methods or components for limiting pressure may be used as the invention is not limited in this respect.

In some embodiments, the pressure may be changed prior to injection. For example, pressure may be increased carefully prior to injection to avoid a sudden increase in pressure associated with the injection process.

In some embodiments, pressures may be selected to be greater than physiological pressure in order to promote transfer of the cellular material to the site of injection. However, pressures should be maintained within ranges that do not damage or otherwise disrupt the cells being transferred. Non-limiting examples of pressures at the site of introduction range from about 5 to about 150 mm Hg. Any suitable intermediate pressure may be used, for example, greater than about 10, 20, 30, 40, or 50 mm Hg, but less than about 75, 100, or 150 mm Hg. In some embodiments, the pressure profile during injection may be a square wave function. (e.g., from about 5-100 mm Hg).

### Pressure Feedback:

In some embodiments, an appropriate pressure profile may be programmed into a delivery device. The appropriate profile may be determined by reference to standard curves or other information (e.g., in the form of databases) that can be used to determine suitable pressures for different target tissues or organs and/or cell types being injected.

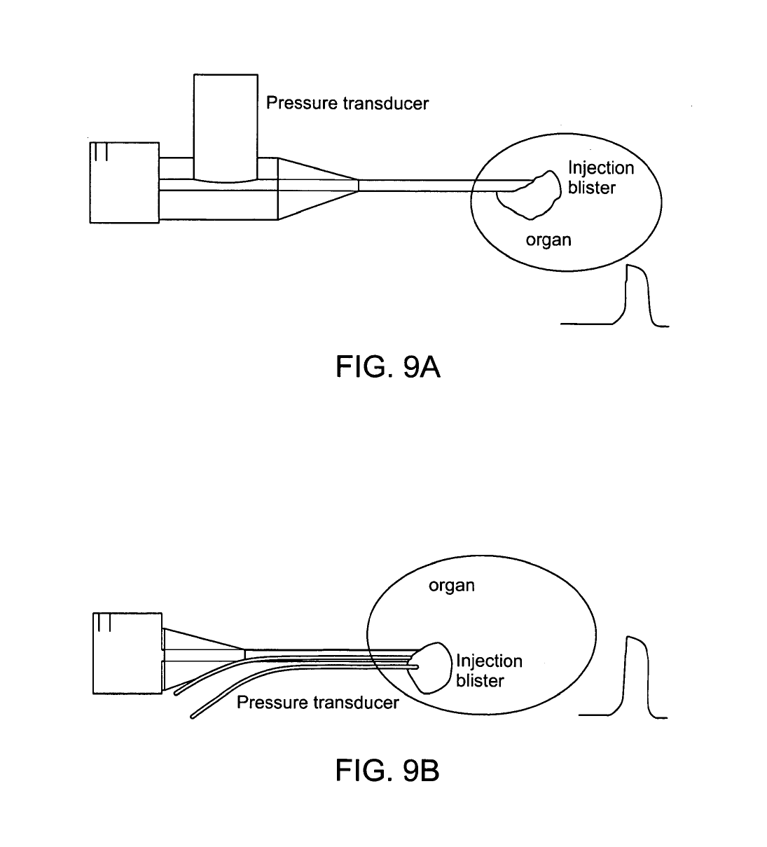

However, in some embodiments, a feedback mechanism may be provided to monitor the pressure during delivery and adjust (e.g., automatically) the pressure exerted by the fluid delivery system. FIG. 9A-9B.

In some embodiments, a pressure transducer may be used to determine the pressure at the site of cell introduction. In some embodiments, the pressure transducer may directly measure the pressure at the site of introduction (e.g., a pressure transducer may be introduced into the blister at the site of injection, either separate from or integrated into the working end of a cell introduction device). In some embodiments, the pressure transducer may indirectly measure the pressure at the site of introduction by measuring the pressure of the cellular preparation at any location within the device (e.g., within any channel, reservoir, or other location that is pressurized in order to cause delivery of the cellular preparation) or by measuring the pressure of the pump or other device that is used to deliver the cellular preparation. It should be appreciated that a pressure measured indirectly may require a standard curve or other correlation to be used in order to determine the pressure at the site of introduction, because the pressure at a location being measured may be higher than the pressure at the introduction site.

Accordingly, in some embodiments one or more pressure transducers may be located at any suitable position on a device. In some embodiments, a pressure transducer may be located near the opening of the working end. In some embodiments, a pressure transducer may be located on the outside of the device (e.g., on the outside of the working end). In some embodiments, a pressure transducer may be located within the chamber or channel of the working end (e.g., within a needle).

For example, a pressure transducer can be connected to the barrel of a needle in some device configurations. Various needle sizes may be used, including, for example, needles having a size in a range of 7 gauge to 33 gauge, e.g., a gauge 28 needle, may be used. In some embodiments, positioning of a pressure transducer in the barrel of the needle allows the transducer to measure the pressure at the injection site while avoiding unnecessary damage in the organ.

In some embodiments, a connector is located at or near the end of a cell introduction device for measuring pressure in the device. Often the connector has a internal diameter that is comparable to the internal diameter of the needle connected to the device. In some embodiments pressure in the connector is similar to pressure in the needle at the injection site. In some embodiments, the pressure reading is used to detect a pressure at the site of injection (e.g., blister) that signifies the acceptance of the injected materials at the injection site (e.g., formation of a blister). In some embodiments, the methods allow the use of a relatively small non destructive needle (e.g., a 28-gauge needle) and still permit measurement of pressure at the injection site. In some embodiments, a pressure reading at a injection site can be fed back via a controller to control a pump (e.g., to control a pump output to inject a specified volume, to output a volume over a predetermined period of time, to output a volume within a predetermined pressure). In some embodiments, the system controls the delivery of a fluid into a tissue in a physiologically acceptable manner and with acceptable spatial control. In some embodiments, the pressure measured by a transducer is feedback to a controller that controls flow of a fluid from a pump such that the tissue accepts the fluid with minimal blowback and good spatial delivery.

In some embodiments, a damaged area of an organ (e.g., an infarct, astrocyte scar, or sclerotic tissue) is harder than undamaged tissue. As a result, when a transdermal patch is placed on damaged area of an organ (e.g., of heart, mylinated nerve, liver, etc.) the damaged area can reaquire a higher pressure for injection.

Accordingly, in some embodiments membrane resistors may be used on a plurality of working end (e.g., needle portals) on an array (e.g., transdermal patch) to allow for fluids to be released only in areas of appropriate pressure corresponding to healthy tissue. In some embodiments, this helps to maximize the delivery of cells or drugs to the healthy sites; inject cells as close to the edge of the healthy and sick cells as possible; and/or to accommodate irregularly shaped damaged areas and maximize the delivery of cells to healthy tissues. Since the diseased tissue will have higher release pressures than the healthy regions, the flow will preferentially target healthy tissue. This can be useful to minimize waste and not depositing valuable live cells into dead areas.

In some embodiments, a lower pressure resistor will be placed in areas of healthy tissue so lower pressures will force fluid through at lower pressures. Higher pressure resistors can be placed in areas that will contact diseased or dead tissue. The high pressure needed to force fluid through to the diseased/dead areas will never be reached. In some embodiments, the pressure is monitored by a pressure feedback circuit to the pump. If the flow starts the pressure will be monitored. In some embodiments, a pressure drop can be detected corresponding to the flow into the healthy areas. The pressure limit at which this occurs can be used to deliver all or part of a sample. Since a higher pressure is not needed, the pressure to open valves in the unhealthy region will not be reached and those valves will not open.

In other embodiments, cells may be introduced at the tissue site at a varying pressure over a delivery time. For example, the cells may be introduced using a pulsatile flow such that the cells are forced into the tissue site and the pressure allowed to decay or otherwise drop before another pressure pulse is applied. Such an approach may allow the tissue to move, separate or otherwise permit the cells to be introduced at the tissue site without requiring pressures or flow rates above what might otherwise be required. In another embodiment, cells may be introduced based on delivered volume. For example, an introduction protocol may call for the introduction of several microliters of cell fluid over a desired delivery time. The pressure, flow rate or other parameters may be adjusted to achieve the desired volume delivery over a specified time. In some embodiments, the flow may be ramped up or down and the flow may be programmed to accommodate the back pressure and resistance characteristic of a tissue at a target injection site (e.g., in order to maximize fluid delivery at a specific location). Accordingly, a system of the invention may be controlled to produce a variable flow rate involving ramping and/or pulsatile flow patterns. In some embodiments, a system may include feed-back loops (e.g., including appropriate sensors and controllers) to respond to environmental (e.g., tissue) back-pressure and adjust to provide the desired force and/or pattern of delivery.

### Configurations for Maintaining the Temperature of a Cell Preparation:

In some aspects, the temperature of a cell preparation is carefully controlled during a cell introduction process. In some embodiments, Applicants have recognized that by maintaining the cells at lower than body temperature or lower than room temperature (e.g., lower than 37, lower than 30, lower than 25, or lower than 20 degrees Celcius), oxygen consumption (and other metabolic processes) can be maintained at lower levels than if the cells were allowed to equilibrate with room temperature or higher (e.g., when loaded into a syringe). A lower metabolic rate (e.g., lower oxygen consumption) protects the cells from the accumulation of waste products and/or from responding to cues that may change their developmental state (e.g., reduce their ability to grow and/or differentiate appropriately in vivo after delivery).

In some embodiments, a cell preparation that is stored in a cooled or frozen state prior to introduction is warmed to a selected temperature before the introduction into a recipient. The selected temperature may be room temperature, body temperature, or any other physiologically compatible temperature. In some embodiments, the rate at which the temperature of a cell preparation is varied (e.g., warmed) is controlled (e.g., to a slow regular rate of temperature change to minimize trauma, cell damage, and/or cell death associated with rapid changes in temperature. In some embodiments, the timing of a change in temperature (e.g., warming) can be important to avoid the cell preparation from being exposed to an inappropriate temperature for an excessive period of time prior to introduction into the recipient. Cellular metabolism can generate waste products that reduce cell viability. Accordingly, a cell preparation maintained at room temperature or body temperature (or other temperature that promotes cellular metabolism) becomes progressively less viable over time. A change in cell viability or function may occur even over the span of a few minutes. Accordingly, in some embodiments, a cooled or frozen cell preparation is warmed to an appropriate temperature immediately prior to introduction (e.g., injection) into a recipient.

It should be appreciated that external and/or internal components may be used for temperature control. In some embodiments, external jackets may be used. In some embodiments, internal elements may be used. It should be appreciated that the components may be coils, Peltier elements, resistors, etc.

In some embodiments, a cell introduction device may include an integrated temperature regulator with heating and/or cooling components that allow the temperature of the contents to be regulated. In some embodiments, the reservoir of a syringe includes a temperature regulator. In some embodiments, the working end of a syringe includes a heating or cooling component. In some embodiment, the heating/cooling component may be a sheath or jacket on the exterior of the device or a portion thereof (e.g., the reservoir). In some embodiments, the heating/cooling component may be within the chamber of the device.

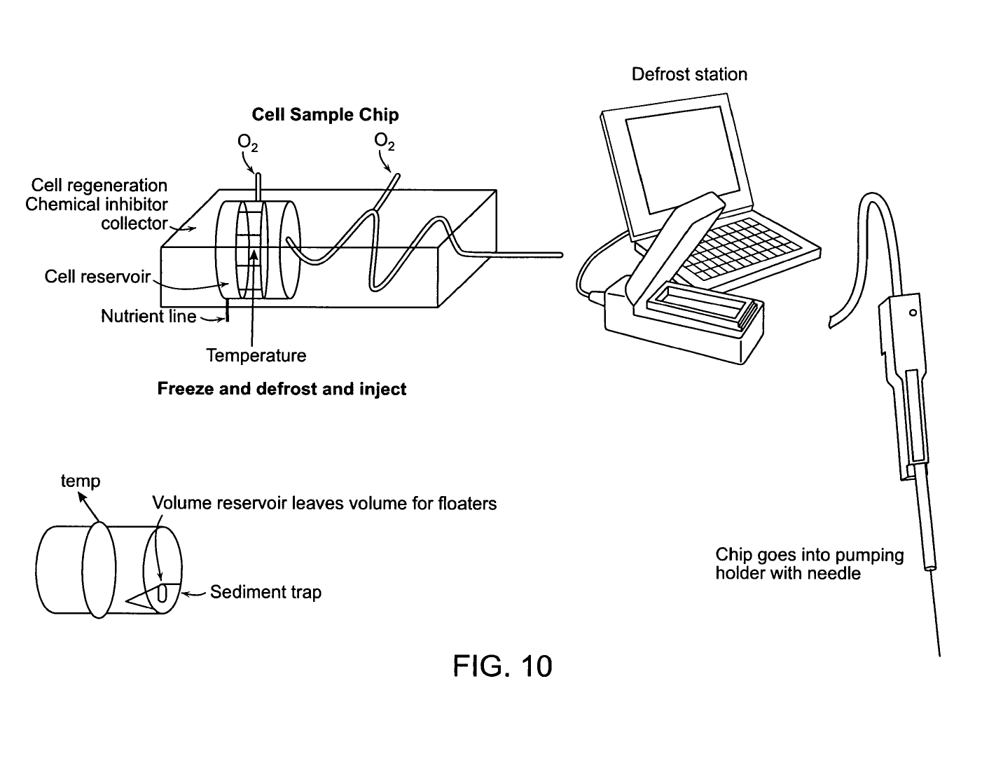

FIG. 10 illustrates a non-limiting embodiment of a defrost system in which a support device (e.g., a chip) containing cells may be stored in a frozen state. The frozen support member may be defrosted in a separate defrost station that controls temperatures and/or temperatures gradients appropriately. The defrosted support device may be stored at an appropriate temperature in the defrost station and then inserted into a cell delivery device for injection into a target site. In this embodiment, the support device also provides one or more support functions (e.g., oxygenation) and one or more filtration functions (e.g., to remove unwanted chemicals and or debris) for use prior to injection. In some embodiments, a support device without any of these functions also may be defrosted using a stand-alone station as described herein. However, it should be appreciated that in some embodiments the defrost function may be provided by the injector and a frozen support device may be placed directly into the injector where it is thawed under controlled conditions prior to use.

In some embodiments, a temperature regulator may be provided that is not integrated with the cell introduction device. For example, the temperature regulator may be a stand-alone cooler/heater that is adapted to receive one or more cell introduction devices and maintain appropriate temperature profiles. In some embodiments, the temperature regulator may include one or more ports shaped to fit one or more portions of a cell introduction device (e.g., the working end and/or reservoir of an injector). In some embodiments, a stand-alone temperature regulator may include a linear or two-dimensional array of ports. In some embodiments, the temperature of all the ports is controlled by the same regulator. In some embodiments, each port is individually regulated or subsets of ports are independently regulated.

In some embodiments, the temperature of a cell introduction device may be maintained and regulated using a removable sheath that is adapted to fit one or more portions of the cell introduction device (e.g., the working end and/or reservoir of an injector), and that contains heating and/or cooling components.

In some embodiments, one or more portions of a cell introduction device is designed to conduct temperature changes rapidly and efficiently. The design may include the material and/or the configuration (e.g., shape, wall thickness, and/or other physical features) that promote efficient heat conductance.

Accordingly, in some embodiments a handling station is provided that can be used to transition cells from a storage temperature to an injection temperature (e.g., to thaw frozen cells). In some embodiments, the handling station is separate from the injector device and can be used to reproducibly control the temperature of a cellular sample prior to loading into an injector device.

However, it should be appreciated that in some embodiments an injector device may include a temperature control element to thaw frozen cells.

In some embodiments, the temperature of a cellular preparation is maintained at about 15 to 20 (e.g., about 18) degrees Celsius after thawing, prior to injection, and/or during injection.

### Configurations for Maintaining the Physiological Environment of a Cell Preparation:

In some embodiments, a device or system of the invention may include one or more sources of nutrients for the cells. For example, a carbon source, oxygen, and/or other nutrients may be supplied via appropriate tubes or lines as described herein. In some embodiments, one or more detectors may be used to evaluate the physiological state of a cell (e.g., using infrared as described herein). In certain embodiments, one or more detectors may be used to evaluate the levels of specific nutrients, toxins, or other physiological parameters (e.g., oxygen, carbon dioxide, pH, glucose, etc., or any combination thereof).

In some embodiments, a material that changes properties in response to an analyte and/or a physiological stimulus (e.g., temperature, oxygen levels, carbon dioxide levels, etc.) may be used to monitor the levels of one or more of these physiological stimuli. In some embodiments, the material may be used to coat a structure that is in contact with a cellular suspension, for example, one or more internal surfaces of a device. In some embodiments, such a material may be used to coat one or both endwalls of a syringe, or a surface of the plunger that comes into contact with the cell preparation. In some embodiments, the material is not used on the side-walls of the syringe or on the sides of the plunger in order to avoid potential leaks due to the presence of the material. However, the material may be used in these locations if it does not result in fluid leaks. In some embodiments, a cell container (e.g., an Eppendorf tube, or a portion thereof) may be coated with a material so that cells introduced to the container can be monitored. Examples of material include polymers (e.g., polymers available from Polestar Technologies, Inc., Needham Heights, Mass., USA). Such polymers can quench light at particular wavelengths, and the degree to which they quench the light signal varies as a function of the level of a particular analyte (e.g., oxygen) in a liquid that contacts the polymer. Accordingly, a device in which an internal polymer coating is being used to detect one or more analytes or physiological stimuli also may include a region that is transparent (e.g., part of the wall may be transparent) for the appropriate light wavelengths so that the polymer can be illuminated from outside the device and the signal from the polymer can be detected outside the device. It should be appreciated that the polymer may be coated on a portion of the device using any suitable technique, for example, it may be polymerized or otherwise deposited, or it may be provided on a membrane that can be attached to the device (e.g., an adhesive membrane). It also should be appreciated that different materials that are responsive to different molecules may be used as aspects of the invention are not limited in this respect. For example, a material (e.g., a polymer) that is responsive to glucose or other metabolite may be used.

### Configurations for Maintaining a Homogeneous Preparation of Cells:

In some embodiments, aspects of the invention relate to methods and devices adapted to maintain a homogeneous cell suspension prior to or during injection and/or prior to freezing and/or storage of a cell preparation. In some embodiments, one or more active or passive mixing components may be incorporated into a device or system of the invention. Accordingly, cell preparations may be mixed using any suitable static or active mixing device. In some embodiments, static cell mixers may be based on a pattern or pathway of physical obstructions or protrusions within the flow pathway of a cell preparation. It should be appreciated that any device described herein (e.g., a cell introduction device, including but not limited to, an array of needles) may include one or more cell mixers (e.g., static cell mixers) within the flow pathway of the cell preparation (e.g., before a manifold, or within each channel of a multichannel device).

In some embodiments, one or more metallic or magnetic elements (e.g., beads, bars, spheres, or any other shape of magnetic element) may be introduced into a cellular suspension (e.g., in a delivery device or in a storage device). The elements can be used to stir or mix the suspension by moving them within the cellular preparation using a one or more magnets on the outside of the device. The magnets on can be moved manually (e.g., it/they can be moved up and down on the outside of a syringe containing magnetic or metallic elements) or its movement can be automated. In some embodiments, one or more magnets may be electromagnets. It should be appreciated that the elements introduced to the cellular preparation may be coated with any suitable coating that does not interact with cells (e.g., a PTFE (polytetrafluoroethylene), for example, available under the brand name Teflon, or other suitable coating.

In some embodiments, the controller of the pump may be used to provide the signals and/or power to automate the mixing process (e.g., by providing suitable electromagnetic stimuli).

In some embodiments, cells in a syringe may be kept in suspension by rotating the syringe while it is positioned in a syringe pump. Accordingly, the syringe pump may include a rotating attachment or stage to which the syringe can be attached. Movement (e.g., rotation around an axis) of the attachment or stage may be motorized or powered using any suitable technique. In some embodiments, a syringe can rotate along its long axis thereby keeping cells mixed and suspended. This can help maintain oxygenation of the cells. In some embodiments, the syringe body may be connected to a gas line to provide air or oxygen to the cells in the syringe. Mixing (e.g., by rotation) also helps deliver reproducible numbers of cells and also allows the number of cells to be determined or predicted more reliably, because a homogeneous cell preparation is maintained thereby avoiding clumping and settling that can give rise to inconsistent cellular injections.

In some embodiments, a device or system may include one or more static flow mixers. In some embodiments, converging or intersecting fluid flows in a device may be used to generate mixing of the fluids in the device. Accordingly, some devices may be designed to include one or more static mixers, and/or one or more fluid flow patterns that result in two or more flows mixing with each other.

In some embodiments, cells may be mixed during freezing, during defrosting, prior to injection, in an injection device, or any combination thereof. In some embodiments, cell preparations are continuously mixed. However, in some embodiments, cell preparations are mixed at regular intervals (e.g., intervals that are known to cause settling or clumping of cells—these intervals may be different for different cell types). In some embodiments, a device may include an alarm or other signal that indicates when the cells should be mixed. The triggering event can be time (e.g., relative to a threshold time after which cells need to be mixed) or based on one or more detectable parameters (e.g., optical density, or other measurement) that is indicative of clumping or settling. Accordingly, in some embodiments a device may include one or more sensors for detecting signal(s) indicative of non-homogeneous cell suspensions.

In some embodiments, information relating to the mixing or other features relating to the homogeneity of a cellular suspension may be maintained on a patient database.

In some embodiments, a cellular preparation may be oxygenated by flowing air or an oxygen containing gas mixture (e.g., oxygen or oxygen mixed with one or more other gases) over the surface of a liquid that contains the cells. Alternatively, or in addition, the cell preparation may be mixed to promote oxygen exchange between the cells and the environment. In some embodiments, a cell preparation may be vortexed (e.g., to form a funnel) in order to maximize the extent to which oxygen can reach all the cells in the preparation. A funnel (e.g., from vortexing) can extend to the lower regions of a cell preparation in a container (e.g., in a tube such as an Eppendorf tube) thereby promoting oxygen exchange throughout the height of the cell preparation. In some embodiments, a cell preparation being vortexed also may be oxygenated (e.g., using a gas conduit that can be attached to the container in such a way that gas can be flowed over the upper surface of the cellular preparation or over the exposed surface of a funnel caused by vortexing).

In some embodiments, a container being vortexed also may be cooled (e.g., to below 37, below 30, below 25, below 20, below 15, below 10 degrees Celsius, or to lower temperatures). This can be useful to reduce oxygen metabolism by the cells in addition to providing oxygenation, thereby minimizing the risk that the cells with lack oxygen. It should be appreciated that vortexing and/or cooling may be performed during and/or after a cell preparation is thawed. A cell preparation may be maintained under continuous vortexing and/or cooling during a process that may involve taking one or more samples from the preparation to introduce to one or more tissue sites.

Accordingly, in some embodiments a device may include a mixing station that provides an orbital or vortexing mixing motion. It should be appreciated that this station may include a motor, a support that provides an appropriate shaking or mixing motion and to which a cell container may be attached. In some embodiments, the mixing station may be temperature-controlled (e.g., using a Peltier element or other suitable heating and/or cooling element). In some embodiments, the mixing station may be in an enclosed space that nonetheless remains accessible through an opening (e.g., covered by a lid). This may allow for more effective or efficient temperature control.

Techniques for Detecting Fluid Loaded into a Device:

In some embodiments, aspects of the invention relate to configurations and methods for determining whether a device (e.g., a syringe) has been loaded with a fluid. In practice, it can be difficult to determine when a fluid is being drawn into a device such as a syringe. For example, if no bubbles are present, there is little contrast to confirm that a fluid is present. Accordingly, it can be difficult even with visible inspection to tell when little or no fluid is drawn into a device, for example, due to a clog (e.g., in a syringe needle), a broken seal, or other defect.

In some embodiments, a device may be configured to allow the internal fluid level to be detected and/or monitored. In some embodiments, one or more detectors (e.g., in the form of a flexible membrane, a patch, or other configuration) may be attached to the outside of the device to determine whether fluid is drawn in. In one non-limiting example, an ultrasonic detector may be used to detect a sound deflection from the interface between the fluid being drawn into the device and the air or other material that is in the device prior to loading the fluid. In another non-limiting example, a capacitance strip may be attached to the outside of a device. It should be appreciated that one or more other appropriate detectors may be attached to the outside of the device. It also should be appreciated that in some embodiments, one or more detectors may be integrated into the device (e.g., into a wall of a syringe). In some embodiments, one or more detectors may be attached to the inside of a device. Regardless of the location and/or number of detectors on a device, the signal from the detector(s) may be processed in any suitable manner. In some embodiments, the level of fluid may be displayed for a user to monitor. In some embodiments, a numerical representation of the fluid level may be provided. In some embodiments, a signal may be generated when the fluid is correctly loaded. In some embodiments, a signal may be generated if the fluid is incorrectly loaded (e.g., insufficient or no fluid is loaded). It should be appreciated that any suitable signal may be used (e.g., visual, audible, or other signal, or any combination thereof). Accordingly, a device may include an alarm that is activated if no or insufficient fluid is loaded.

In some embodiments, a device may include a pattern (e.g., etched or otherwise displayed within a transparent portion of the device, for example printed on the wall of a glass portion of a syringe body) that changes upon exposure to fluid. For example, the clarity or brightness of the pattern may change detectably upon exposure to fluid. In some embodiments, the pattern is a colored pattern. However, in some embodiments, a pattern is a grooving, etching, or other physical alteration of part of the device. It should be appreciated that these or other configurations may be used for detecting wetting when filling a needle (e.g., to determine whether it is full or not).

In some embodiments, a refractive index lens or other magnifying element may be incorporated into a device (e.g., into the glass of a syringe). In some embodiments, an asymmetrical shape of at least a portion of a device, or other physical shape (e.g., bulge, protrusion, etc.) within a transparent portion of a device (e.g., within a syringe portion) may be used to magnify the contents of a portion of the device to help detect fluid levels.

Methods of Loading a Device without Generating Bubbles:

In some embodiments, it is desirable to avoid getting bubbles into the lines of a device or into a syringe barrel. Bubbles can be difficult to clear, particularly for small volumes (e.g., for <1 mm internal diameter syringe barrels).

In some embodiments, bubbles can be avoided by moving a plunger sufficiently slowly to not cause a vacuum. Since an empty syringe is an air column and highly compressible, the plunger should be drawn slowly enough to build up enough pressure to move the liquid volume in perfect synchronization with the plunger. This allows the barrel of the syringe fills without cavitations or without forming bubbles.

In some embodiments, it is easier to obtain an accurate fluid withdrawal without forming bubbles if the inside of the syringe needle and barrel are wetted. When there is only air in the internal volume, the wet surfaces on the inside of the syringe or barrel walls provide a hydraulic advantage when the plunger is withdrawn and this results in a non-compressible medium pulling up the liquid.

Accordingly, in some embodiments, the invention provides a procedure and automated process for loading a solution into the syringe automatically. In some embodiments, in a first step, a syringe plunger is pushed all the way into the syringe, the syringe is placed in a pump, and liquid tubing is connected to the syringe. In a non-limiting embodiment, to fill the tubing with solution to the end of the line, the syringe plunger is repeatedly pulled back and pushed out, with each cycle bringing more fluid into the syringe. In one embodiment, the syringe plunger is pulled back to no less than 5% of the volume of the syringe. The fluid is pushed back out and then drawn back in to 3% of the volume of the syringe. This is repeated to 1% and the syringe is then returned to the empty start position. Subsequently, the syringe is loaded by withdrawing a fluid into the syringe using a flow rate no greater than 10% of the rate of expected delivery (this is done until the syringe is full).

In a further step, using a flow rate no less than 2 times the desired flow rate of the expected infusion, a fluid is withdrawn in no less than three pulses. The plunger can then be withdrawn to the fullest extent and the syringe is now full.

It should be appreciated that this process may be automated to produce a loaded syringe with no bubbles.

### Fluid Delivery:

In some embodiments, fluid in the device may be delivered using any suitable apparatus or technique, for example, a mechanical, a pneumatic, a hydraulic, or a combination system or technique. In some embodiments, a fluid-driving mechanism (e.g., a pump) may be remote from the site of delivery. In some embodiments, the controller and/or fluid driving mechanism may be separate from the introduction device, and connected only via a wire, a tube, or a combination thereof. Accordingly, the mass associated with the working end of the device can be minimized. In some embodiments, a device can have a zero dead volume. For example, the volume of a needle at the tip of the working end can be part of the delivery device volume. Accordingly, in some embodiments a device can deliver a zero dead volume injection or a series of injections from a volume that is the same as the volume of the tube connected to the driving mechanism and the needle.

In some embodiments, a tubular portion (e.g., a rigid or flexible tubular portion) of a device may coiled or otherwise arranged (e.g., in an irregular or regular pattern or shape, for example a coil, a spiral, a serpentine or other pattern or shape). This portion may be placed in an environment that allows for temperature control (e.g., a temperature control box or other device). In some embodiments, the tubing may be arranged (e.g., coiled) into the box or other device and heat or cold is applied to provide temperature-control for the flow or contents of the tubing. In some embodiments, this technique may be used to heat or cool a fluid moved by a peristaltic pump, a syringe, or any other fluid-moving device. In some embodiments, the tubing may be pre-filled, and optionally assembled with a needle or other tip. In some embodiments, the tubing maybe environmentally controlled for pressure, temperature (e.g., frozen or heated), oxygen, chemical content (e.g., using one or more chemical absorbers) or any combination thereof. In some embodiments, a tube described herein may be connected to a reservoir or other volume to provide for multiple delivery volumes (e.g., multiple injections).

It should be appreciated that a system wherein the working end is separated from heavier items such as a pump and/or controller may provide several benefits, including being lightweight, easy to handle, easy to mount on a micro-device. In some embodiments, a zero dead volume device provides for a reproducible injection volume. In some embodiments, cross-contamination of cells can be reduced or avoided by using a hydraulic or pneumatic delivery force. In some embodiments, one or more of the tubing, tip, and/or other components are easy to freeze for storage, and/or environmentally control for delivery, or a combination thereof.

Accordingly, it should be appreciated that any form of pump or actuator may be used either to directly generate pressure on a fluid and drive it through a device or to move a plunger or other physical element that drives the fluid.

### Configurations for Adding Additional Components:

In some embodiments, a cell introduction device also may include a feature for introducing desirable molecules to a cell preparation prior to injection.

In some embodiments, the cells may be introduced at the tissue site with one or more materials to potentially enhance the effect of the cells. For example, the cell fluid may include a material to absorb or otherwise reduce an effect of any toxins in the cell fluid, a material to aid in adherence of cells at the tissue site, a material to aid in growth or survival of cells, a material to stimulate or otherwise aid in cell division, adhesion or penetration into the tissue, a material to protect the cells from a host response (e.g., an anti-inflammatory and/or immunosuppressive material), a material to provide physical support to cells at the tissue site, and/or a material to aid in imaging of cells at the tissue site. For example, a material may be added to the cell fluid (whether in a syringe reservoir, at a reservoir on a patch or other support, and/or directly into the patient) that absorbs or otherwise neutralizes the effect of cell signaling molecules that cause progenitor cells to differentiate into unwanted cell types. As a result, undifferentiated cells may remain in a desired state until the cells are introduced at a tissue site. Cell signaling molecules and/or other materials may be removed by dialysis, solid phase extraction, antibody binding, and/or other techniques. Materials may be added directly to the cell fluid, and/or may be added separately whether via the working end or another device. In one example, a plurality of beads that tend to remain near at least some of the cells may be introduced with the cells at the tissue site. In some embodiments, the beads may be arranged to provide a particular function, such as toxin absorbance, yet be retained in the syringe or other cell introduction device after the cells are introduced to the tissue site. The beads may perform at least one of the following functions: enhance imaging of the tissue site, be resorbable (e.g., include a fibrin, PLA or other material), include an oxygen source for cells, include a growth factor for cells, and/or include a toxin absorber. In another example, a material may be added to the cell fluid that includes an imaging contrast agent, e.g., a rare earth metal such as europium to help determine via imaging where the cells are located in the tissue. In another embodiment, cells may be introduced at the tissue site while contained inside of one or more capsules. The capsules may help isolate the cells from potentially harmful environmental conditions, such as excessive shear stress, heat, cold, toxins, and so on. The capsules may be made of a resorbable or other degradable material (e.g., gel, gelatin, polymer, etc.) such that the capsules open after the cells are introduced at the tissue site.

In another aspect of the invention, a solution may be introduced at a tissue site before cells are introduced at the site. The solution may provide a variety of different functions, such as enhancing cell adhesion at the site, providing nutrients for the cells, reducing shear stress and/or pressure during cell introduction, assisting in oxygenating the cells, helping to reduce toxins at the site, providing growth factors, providing a scaffold or other physical support or structure for the cells, and others. For example, a fluid or gel containing beads, fibers or other physical structures may be introduced at the tissue site before the cells. For example, a material that can be injected at a high enough physical strength to open the tissue at the tissue site, but whose strength can then be decreased (or decreases on its own) to ease the subsequent injection of cells could be used. In one embodiment, such a material may be a gel that partially melts at body temperature but that is injected at a temperature below body temperature, or a fluid that exhibits thixotropic properties or can be made less viscous upon the application of external energy, such as heat, UV radiation, ultrasound, etc. The beads, fibers, etc. may provide a relatively porous structure into which the cells may move and/or may provide physical support to the cells at the site. Such solutions may alternatively or additionally be provided after the cells have been introduced at the tissue site.

### Implantable Devices and Configurations:

In some embodiments, the tip of an injector (e.g., the tip of a needle-like device) may be left at the site of injection to form a protective capsule. For example, the needle-like device, or a portion thereof, may include a tip region that is detachable (e.g., broken off at the site of injection, or remotely detachable using an appropriate release mechanism, or using any other suitable technique or configuration) and that can be left at the site of injection. In some embodiments, the tip region is resorbable or biodegradable. In some embodiments, the tip region is porous. In some embodiments, the tip region is sealed. In some embodiments, the tip region is open to allow migration of the cells and/or transport (e.g., by diffusion) of nutrients, oxygen, waste, etc., or any combination thereof.

In another embodiment, the working ends themselves and/or a reservoir on the support may contain cells that are introduced to the tissue site by diffusion, osmosis, or other mechanism. The working ends and/or the support may be made of a resorbable material, e.g., in the form of a patch that persists at the tissue site long enough to introduce cells, but later degrades. The support may be flexible, allowing the support to conform to a tissue surface, and/or allowing the support to move with a moving tissue surface. Flexibility of the support may also allow the support to be rolled or otherwise reduced in size so the support and working ends can be deployed through a catheter or other device in a minimally invasive surgical technique. The support and/or working ends may include a gel, adhesive or other material that helps to keep the support and working ends in place at the tissue.

In some aspects of the invention, one or more working ends described herein (e.g., in any of the examples, figures, or description herein) may be connected to a pump that is remotely controlled (e.g., wirelessly controlled) and/or programmed to operate independently and/or in response to one or more input signals (e.g., from one or more sensors on an injector system). In some embodiments, an injector system that includes a pump may be implanted into a subject to deliver cells over a period of time. In some embodiments, the implanted injector also includes a power supply such as a battery or other power source. In some embodiments, one or more components of the system may be moldable and/or shaped to fit in or on the tissue site of interest.

In some embodiments, a system or device (e.g., an implantable device) may contain a sufficient volume to deliver an appropriate amount of cells over a predetermined time period. In some embodiments, a system or device (e.g., an implantable device) may include a reservoir. The reservoir may have an internal void volume (e.g., that can be filled with a cell preparation) of between 50 to 500 microliters, e.g., from 50-100, 100-200, or 50-250 microliters. However, a system or device may have a smaller or larger reservoir volume depending on the applications. In some embodiments, a cell preparation may be released or injected in a continuous flow. In some embodiments, the cell preparation may be released or injected in incremental amounts (e.g., 5-10 microliter amounts) at regular time intervals (e.g., at time intervals of 1, 2, 3, 4, 5, 5-10, 10-20, 20-30 minutes, or longer time intervals).

Accordingly, in some embodiments, a system of the invention comprises a delivery device that does not require cellular injection per se. Rather cells are introduced into a delivery chamber. In some embodiments, appropriate pressure, temperature, and other parameters are measured and/or controlled as the cells are introduced into the chamber. The chamber is then introduced to the target site and left in place. In some embodiments, the chamber is resorbable. In some embodiments, the chamber is porous and biodegradable so that the cells can survive during the period of resorption. In some embodiments, a working end or tip portion thereof may be ejectable, breakable, or otherwise detachable at the site of injection. In some embodiments, one or both ends of a cylindrical chamber are sealed (e.g., by a resorbable material). In some embodiments, one or both ends remain open to allow exchange of oxygen, nutrients, metabolites, and waste material, e.g., for a period during which the chamber walls are resorbed.

In some embodiments, a working end or other tubular structure may include an outer shaft or wall within which an inner cylindrical core structure can move. Cells may be contained within the inner core structure. In operation, the working end is used to introduce the inner core to the target site. As the working end is withdrawn, the inner core structure is extruded from the working end shaft and remains at the site of injection. In some embodiments, this process may involve pressure to remove the inner core. In some embodiments, a mechanical actuator may extrude the inner core (e.g., a plunger or other device may be used). In some embodiments, the inner core may be a walled cylinder containing cells. The cylinder may be resorbable. In some embodiments, the cylinder walls may be porous. In some embodiments, the walls may form a mesh that protects those cells from one or more damaging conditions (e.g., excessive pressure or other damaging conditions) at the target site during the introduction process. However, the mesh may allow the cells to migrate into the surrounding tissue after introduction. In some embodiments, the mesh is resorbable. In some embodiments, the exclusion characteristics of the mesh do not prevent cells from migrating out of the core into surrounding tissue. In some embodiments, the core may not include an outer wall that surrounds an inner material. Rather, the core may be a matrix (e.g., a porous matrix, with a regular or irregular structure) that can be deposited at the site of introduction. The matrix may provide structural support for the cells during and immediately after the introduction process. In some embodiments, the matrix may be resorbable. In some embodiments, the material of the matrix may support cell growth as it degrades. In some embodiments, the pores of the matrix may be sufficiently large to allow cells to migrate out from the matrix into the surrounding tissue. In some embodiments, an inner cylindrical core may be less rigid than an injector working end. For example, the core may be flexible, compressible, or otherwise deformable. However, it should be appreciated that the inner core may provide support and protection for the cells being introduced (e.g., by protecting the cells from excessive pressure at the site of introduction) even if the material has less structural rigidity than the injector working end.

### Integrated Devices:

In another aspect of the invention, various techniques may be employed to help enhance the viability or other effectiveness of cells after being introduced at a target site, e.g., a tissue site. In one embodiment, the cell introduction device may include various devices or materials to oxygenate cells, control the temperature of cells, feed the cells, control pH, and so on. For example, while cells are being held before deployment, a circulatory system (e.g., similar to a dialysis system having a suitable membrane barrier between a circulatory fluid and the cell fluid) may provide nutrients, oxygen, and/or other materials to provide a suitable environment for cells to remain alive while awaiting introduction at a tissue site.

In some aspects, a system comprises a miniaturized injector that can inject cells into tissue. In some embodiments, an injector (e.g., a handheld injector) contains a “life support” system for the cells that can keep the cells alive and healthy until they are injected. In some embodiments, a critical period for cell survival is the time between defrosting from long term storage of the cells through injection into the recipient tissue. In some embodiments, a device includes a temperature regulated component that can serve as a controlled defrost station where frozen cells (e.g., in an Eppendorf tube or other container) can be rapidly and controllably brought to the correct temperature. In some embodiments, a target temperature is body temperature. In some embodiments, a target temperature is several degrees cooler than body temperature (e.g., 1-10, 3-5, about 5, or 5-10 degrees centigrade cooler than body temperature). However, cooler or warmer temperatures also may be used. Maintaining the cells at a sufficiently cool temperature is expected to slow or maintain a relatively slow cellular metabolism, thereby increasing the survival rate and/or time of the cells prior to injection.

In some embodiments, maintaining cells at a relatively low metabolic state (e.g., by keeping them cool prior to injection) allows a relatively higher concentration of cells to be used in a preparation for injection. This allows a smaller volume to be injected, thereby reducing damage to the recipient tissue. Currently, cells are held in a syringe for at least several minutes prior to and during the injection during which many cells are expected to die, thereby reducing the viability of the graft. One way researchers and physicians compensate for this is to dilute the cells so that there is a high ratio of media to cells. This has the negative effect of a higher fluid volume being injected into the tissue, resulting in higher tissue damage. The tissue damage promotes cellular defense and repair mechanisms that can kill more of the cells in the injection. Accordingly, aspects of the invention are useful to reduce the degree of host response and enhance the viability of the injected cells.

In some embodiments, a physiological support system (e.g., a miniaturized cell life support system) may be provided to promote and/or maintain cell viability prior to injection. In certain embodiments, the physiological support system may have a size on the order of a pencil or Hamilton 100 microliter syringe. In some embodiments, a system may include a miniaturized fluid circuit containing a small but sufficient volume of cells/media mix to support the cells prior to injection. In some embodiments, the volume is between 10-500 microliters (e.g., 10-250, or 10-100, or about 50 microliters). In some embodiments, the fluid can be circulated within the microfluidic circuit by a mini pump within a circuit including a physiological support component. In some embodiments, the physiological support component is an oxygenation path (e.g., a gas permeable membrane over part of all of the microfluidic circuit which would be supplied with oxygen and/or carbon dioxide).

It should be appreciated that a microfluidic circuit can be incorporated into any suitable support medium. In some embodiments, the microfluidic circuit can be integrated on a flexible plastic material. This may be shaped to fit into any suitable device configuration (e.g., it may be rolled and inserted into the tubular body of a device casing). This configuration provides a large surface area for gas exchange (e.g., for greater exposure to oxygen). This configuration also provides a large total fluid volume in the microfluidic circuit. In some embodiments, the casing can be temperature controlled (e.g., heated and/or cooled). In some embodiments, the injection working end also may be heated and/or cooled.

In operation, the cell/media combination can circulate in the microfluidic chip for as long as needed until the working end is inserted into the patient at which point a valve is opened and the cell/media combination is pumped into the patient.

A microfludic chip can contain additional or alternative physiological support components and/or other components that are useful to prepare cells for injection. In some embodiments, a chip can contain one or more components for sorting the cells to ensure that only healthy cells are actually injected. For example, dead or dying cells could be sorted and removed (e.g., sent to waste) whereas healthy cells are isolated for injection. In some embodiments, a microfluidic circuit can include a filter to remove cellular debris (e.g., a size exclusion medium that lets larger objects pass but catches smaller objects such as cellular debris.

In some embodiments, a cell sorting and/or concentrating mechanism can be used to concentrate cells immediately prior to the injection. In certain embodiments, the concentration rate can be matched to the infusion rate such that only highly concentrated cells are injected.

In some embodiments, a microfluidic chip can contain sensors such as for pH, lactate, glucose, pO₂, etc., or any combination thereof that could indicate cell viability or health and could be used to alter the injection parameters (e.g., including a threshold for a go/no go decision on injection) on the basis of some or all of these parameters.

In some embodiments, one or more of the sensors or other components of a microfludic chip can be in wireless communication with a remote system to maintain the sterility of the microfluidic chip.

As described herein, a working end or other tubular structure used in connection with the microfluidic device may be relatively short (e.g., on the order of 1-5 mm or shorter). In some embodiments, this reduces the time the cells are exposed to an unoxygenated state (as they travel down the working end from the oxygenated microfluidic circuit to the tissue) and promotes their viability and functionality.

In another aspect of the invention, the viability of cells may be assessed before the cells are introduced at the tissue site. For example, various cell characteristics may be assessed, such as heat output from the cells, ATP levels in the cells, oxygen take up/carbon dioxide output of the cells, Na/K pump efficiency of the cells, and/or other characteristics that provide an indication of the cells ability to survive at the tissue site. In some embodiments, infrared profiles of the cells are obtained and evaluated (e.g., by comparison to a standard curve). Unhealthy or otherwise unfit cells may be removed from the cell population that is later introduced at a tissue site. In addition, cells may be assessed for type or other characteristics, and separated as suitable prior to introduction at a tissue site. For example, stem cells that are more suitable for introduction at a heart tissue site may be separated from other cells less suitable for such an application, and the separated stem cells introduced at the heart tissue site.

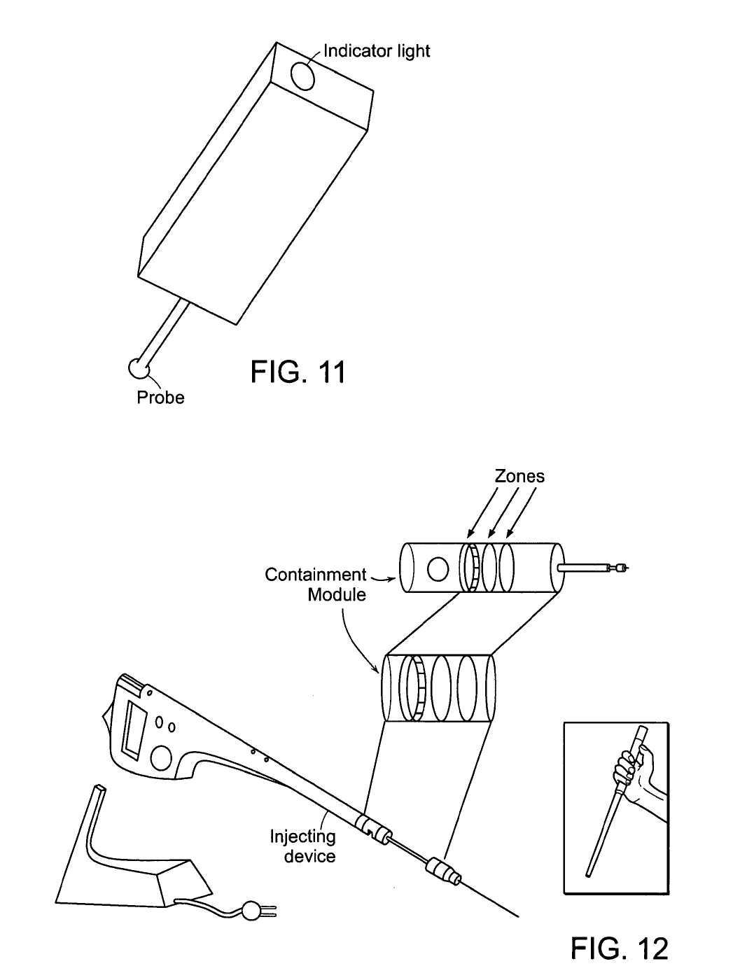

In another aspect of the invention, the cell introduction device may include a device (whether integral to the cell introduction portion or separate) for identifying a suitable tissue site for introducing cells. In one embodiment, a potential tissue site could be imaged (e.g., by a visible light, infrared light, or other technique to detect any informative signal, for example, a chemical and/or temperature signal) and the image data assessed to identify a candidate tissue site. For example, an infrared image of a tissue may reveal areas of cooler tissue (indicating dead or dying cells), suggesting that cells should be introduced in areas around the dead or dying tissue area. In another embodiment, movement of cells, e.g., those of a heart, may be imaged, with cells moving less robustly being identified as dead or dying tissue. FIG. 11 shows a tool that may be used to identify tissue sites in one illustrative embodiment. In this embodiment, a probe may physically contact tissue and assess electrical current in a cell, either in response to electrical stimulation or otherwise. Based on the current level, the device may provide an indication, whether visual and/or audible, for dead/dying cells or live cells. Using this information, one or more tissue sites may be identified, e.g., tissue areas that are near dead/dying tissue but in live tissue areas suitable to support the life of the newly introduced cells. Although current detection is provided as one example for detecting cell viability, other characteristics, such as voltage, resistance, capacitance and/or inductance, may be used in addition to, or in replacement of, a current level. Other assessment techniques may be used to assess the tissue at candidate tissue sites, such as monophasic action potentials, ECG levels, and others. Cells being injected and/or tissue at a target site may be evaluated using one or more parameters (e.g., pressure, temperature, vibration, color, quantitative or qualitative chemical properties, electrical stimulation, etc., or any combination thereof).

### Cartridges:

FIG. 12 illustrates a non-limiting embodiment of a support device also referred to as a containment module. In some embodiments, a pumping module also may be included. In some embodiments, this module may be a cartridge or bullet-like or microcircuit type device, that may or may not be used to store samples for freezing and/or defrosting. In some embodiments, cells may be defrosted in the module and transferred to the injecting device. Accordingly, such modules can be used in the delivery device itself or in the separate defrosting unit. It should be appreciated that such a module may be flexible or rigid. In some embodiments, cells are added to the module and frozen for storage and then defrosted in the module (e.g., in a stand-alone station or in an injector, either of which may be adapted to receive the module). It should be appreciated that one or more drugs also may be included in the module (e.g., with or without cells) for injection into a subject. In some embodiments, such modules may include information relating to a patient identity, a technician identity, a cell line being deliver, date of freezing, date of defrost, metrics on measurements made to assure cell viability, survival controls (e.g., O₂, cooling technology, temperature maintained, O₂ level of cells, toxin or filter debris), other identifiers, etc., or any combination thereof. Accordingly, in some embodiments a module may contain all the instructions required for preparation and delivery that can be communicated directly to a delivery system (e.g., injector) when the module is placed there. In some embodiments, a security system can intercede with these instructions to take over manual control (e.g., without erasing the data in the module). The module is illustrated with one or more zones (e.g., for filtering or other processing). A module also may include one or more controllers and/or circuits (and associated power supplies in some embodiments). These features are described in more detail herein. However, in some embodiments a module does not have any such zones or controls or circuits.

It should be appreciated that a cartridge may be of any suitable size or shape. In some embodiments, a cartridge is shaped and/or includes one or more structural features that are adapted to fit into a receiving station in a device. Accordingly, a cartridge may in some embodiments be designed to be a disposable unit that can be used with one or more devices described herein. A cartridge may be cylindrical, ovoid, rectangular, or any other shape. The volume of the cartridge should be sufficient to support the different functional components. Accordingly, a cartridge may be from about several millimeters to about several centimeters long (e.g., 5 mm to about 10 cm, about 1 cm to about 5 cm, about 1, 2, 3, 4, or 5 cm, or smaller or larger depending on the application) in any linear dimension or in diameter, depending on the shape of the cartridge.

It should be appreciated that different aspects of the invention described herein may be used to deliver a small volume (e.g., on the order of 1-5 microliters per injection) to a relatively larger volume (e.g., on the order of 1-5 milliliters per injection) depending on the application. Also, in some embodiments several rounds of injection may be performed at a site. Accordingly, a device (e.g., a cartridge) may be configured to have a reservoir that is sufficiently large to allow for several consecutive injections. However, different sizes of cartridges or reservoirs may be used for different applications.

In some embodiments, a module or other cell container may have structural features (e.g., fins or other structures) that promote heat exchange and can be configured to obtain optimal heat gradients to minimize damage to cells during either of the freezing or thawing processes.

In some embodiments, a cartridge may include a membrane or valve through which cells can be removed, for example for delivery and/or processing. In some embodiments, a cartridge includes a cap that is reversibly attached (e.g., via a screw, clip, or other mechanism) to a portion of the cartridge. In some embodiments, a cartridge includes one or more electrical and or fluid ports that can be used to connect the cartridge to a device such as an injector. In some embodiments, the cartridge includes one or more physical elements (e.g., grooves, depressions, ridges, or other recessed or protruding parts) that can mate with complementary elements on the device, thereby allowing the cartridge to snap into a predetermined position on the device. It should be appreciated that in some embodiments, a cartridge may provide all of the functions that it requires to prepare the cells, and the device provides a conduit or other channel for removing a volume of cell preparation from the cartridge and delivering it to a site (e.g., a tissue site). However, in some embodiments, the support device may provide one or more filtration, mixing and/or other functions.

### Systems:

According to some aspects of the invention, one or more of the following components is integrated into a system for introducing cells into a recipient: a component for maintaining a viable cellular environment prior to introducing cells into a recipient; a component for protecting cells from physical and/or chemical damage during introduction into a recipient; a component for protecting cells from physical, chemical, and/or biological harm after introduction into a recipient; a component for monitoring one or more parameters of the cellular environment prior to introduction, during introduction, and/or after introduction of cells into a recipient, and a controller which may include a microprocessor and/or any other data processing device, one or more volatile or non-volatile memories, communication devices, one or more sensors to detect parameters used to control operation of the cell introduction system, and other components needed to provide desired control and other functions.

In some embodiments, the controller controls one or more components of the cell introduction system based, at least in part, on measurements obtained from the monitoring component. In some embodiments, the controller functions to maintain appropriate temperature, oxygen saturation, pH levels, and/or cellular homogeneity based on measurements of the temperature, oxygen saturation, pH, and/or cellular homogeneity of a cell suspension prior to introduction. In some embodiments, the controller functions to maintain a flow rate that minimizes shear stress on cells passing through a cell introduction device (e.g., cell passing through the working end of the device) based on measurements of the flow rate of the fluid passing through the device. In some embodiments, the controller functions to minimize recipient tissue damage and/or to provide support for the cells after introduction based on measurements of the metabolic activity, temperature, oxygen saturation, and/or pH in the tissue of the recipient at or near the introduction site. In some embodiments, the metabolic activity, temperature, oxygen saturation, and/or pH are assessed using imaging, e.g., infrared imaging. Thus, in some embodiments, the cell introduction system further comprises an imaging component.

It should be appreciated that any appropriate component disclosed herein, or otherwise known in the art, may be integrated into the cell introduction system. Moreover, it should be appreciated that components of the system may or may not be assembled and/or constructed together as a single unit. In some embodiments, the cell introduction system comprises a single power source that provides power to each and every component of the system. In other embodiments, the cell introduction system comprises one or more power sources that provide power to one or more components of the system.

In some embodiments, a system of the invention may include a computer or other processor that can store and/or download one or more parameters of the process, prior to, during, and/or after injection (e.g., cell temperatures, pressures, injection time, etc., or any other parameters referred to herein). In some embodiments, an cell delivery device may include a memory and/or processor and store information relating to the procedure (including, for example, information from a cartridge or module containing the cells, the thawing process, the time, the identity of the patient, etc., the defrosting temperature, pump flow rates, delivery time, volume, flow rate, speed, force, electrical activity, etc., or any combination thereof). In some embodiments, this information can be stored in any suitable form (e.g., in RAM) in the device and then be available to download onto a computer system for further processing and/or storage (e.g., with the patient records) when the device is synchronized (e.g., via a docking port or other wired or wireless connection) with the computer (e.g., during or after the procedure is finished). In some embodiments, a cell delivery device may be self-calibrating (e.g., for GMP compliance). In some embodiments, information about the calibration (e.g., calibration results) also is captured and stored by the device (at least temporarily).

In a non-limiting embodiment illustrated in FIG. 2, operation of the pump 4 may be controlled by a controller 5, which may include a microprocessor and/or any other data processing device, one or more volatile or non-volatile memories, communication devices, one or more sensors to detect parameters used to control operation of the cell introduction device, and other components needed to provide desired control and other functions. Some of the sensors that may be used by the controller 5 include a pressure sensor to detect pressure at the working end (a pressure indicative of a pressure at the working end may actually be sensed upstream of the working end), a temperature sensor to detect a temperature of fluid at the working end 1 or elsewhere in the device, an oxygen sensor to detect oxygen concentration of fluid associated with the cells, a position sensor to detect a position of the working end relative to a tissue site, as well as sensors to detect a flow rate of cells introduced to the tissue site, a force used to insert the working end into a tissue, a rate of travel of the working end, a penetration depth of the working end into tissue, a penetration time of the working end in the tissue, a shear stress on cells (including shear stress experienced in a syringe body or other reservoir, in a conduit to the working end, at the working end and/or at the tissue site), and/or resistance of the working end to a rotational force on the working end. It should be understood that sensors said herein to detect a particular parameter need not actually detect that parameter, but rather may detect one or more parameters that are indicative of another parameter. For example, a measure of flow rate and pressure at the working end may be used to determine a shear stress on cells.

Thus, the controller 5 may control the release of cells from the at least one opening based one or more parameters, including those measured by a sensor, input by a user, or otherwise determined. Depending on the parameter(s) used to control cell introduction, the controller 5 may include one or more actuators or other devices to adjust operation of the cell introduction device. For example, the controller 5 could include a pressure sensor that detects the pressure of the cell fluid (e.g., a liquid mixed with cells to be introduced at the tissue site) or a pressure that is indicative of pressure of the cell fluid at the working end. In some cases, the controller 5 may limit the pressure of the cell fluid to a maximum, e.g., to help improve survival of cells after introduction at the tissue site. In other embodiments, the controller 5 may limit a level of shear stress experienced by cells, and to do so may maintain a flowrate of cells at the working end 1 below a desired level. In other embodiments, the controller 5 may include a heater (e.g., a jacket-type electrical resistance heater in the case of a syringe-type cell introduction device) and/or cooling device (e.g., a heat exchanger with a circulating fluid, a Peltier device, or other) to maintain cells at a desired temperature whether at the working end, in a reservoir of the cell introduction device, and/or at the tissue site.

In another aspect of the invention, a cell introduction device (such as a manually-operated syringe type device) may be pre-loaded with cells ready for introduction at a tissue site in an emergency situation. This type of device could be prepared in advance and used, e.g., in the case of heart attack, without requiring that a patient's own cells be harvested and used to provide cells for introduction. This type of device may be used in conjunction with a system of the invention.

In another aspect of the invention, a cooling homeothermic blanket may be used in conjunction with a cell introduction device or system so as to reduce breathing, heart rate and/or metabolic rate of the patient. This treatment may reduce bleeding and decrease cell death in the patient.

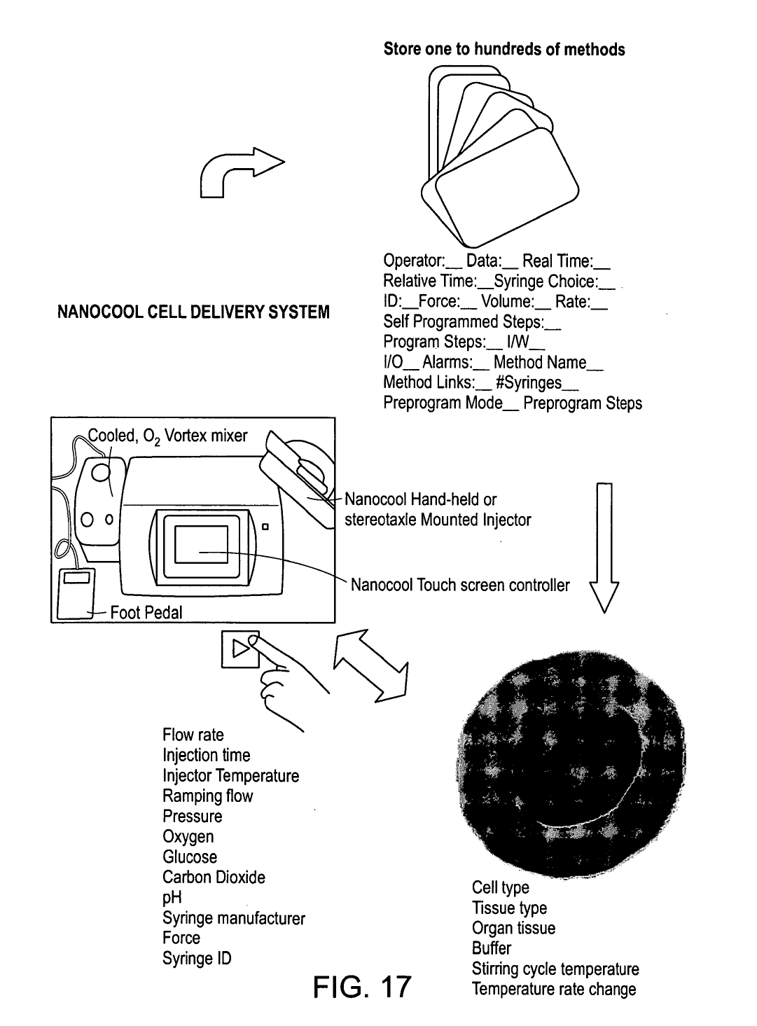

In some embodiments, aspects of the invention relate to a stand-alone apparatus that houses several components described herein. In some embodiments, each component is controlled from the same user interface and/or powered from the same power source. The apparatus may include one or more inputs for receiving information from one or more detectors described herein. In some embodiments, one or more detectors are integrated into the same apparatus housing and/or connected to it via a wire, tube, or other connector (e.g., a rigid or flexible connector). The apparatus may include one or more mixers, heaters, coolers, pumps, actuators, light or other energy sources, or other functional components, or any combination thereof. In some embodiments, one or more of the functional components are integrated into the same apparatus housing and/or connected to it via a wire, tube, or other connector (e.g., a rigid or flexible connector). Accordingly, in some embodiments, an apparatus may include a cooled vortexer (e.g., FIG. 17.), and an injector that are both connected to the same power supply and controlled by the same user interface. Accordingly, a user needs only to switch on one device to activate two or more components as described herein, each of which can be controlled and/or programmed from the same user interface.

In some embodiments, aspects of the invention relate to a method or algorithm that controls and integrates two or more components described herein (e.g., through a single user interface, without requiring separate controls for each individual component). For example, one or more of the follow acts may be automated and/or implemented using an algorithm that can be customized and/or activated through a single user interface: a pump may automatically respond to a pressure feedback loop by altering the pump pressure, a cell storage device may automatically alter the physiological environment (e.g., using pumps and conduits providing different molecules) in response to feedback about the physiological status of the cells or the levels of one or more nutrients and/or waste products; and an injector may automatically deliver a cellular preparation after it has been appropriate processed (e.g., based on one or more detector feedbacks or based on the completion of a predetermined algorithm, for example, implementing a series of temperature changes and/or filtration steps to prepare an appropriate thawed cell preparation).

It should be appreciated that information relating to different steps and or feedback information may be displayed, for example, on the user interface. It also should be appreciated that in some embodiments, one or more wireless connections may be used to convey information or instructions between a controller and one or more other components (e.g., detectors, pumps, mixers, temperature regulators, light or other energy sources, etc., or any combination thereof) or between individual components.

### Evaluating the Tissue Site:

In some embodiments, aspects of the invention relate to detecting one or more physical or physiological features of a target tissue or organ in order to assist in the targeting of a surgical or therapeutic intervention (e.g., a cellular injection). As described herein, in some embodiments a cellular injection may be targeted to one or more zones surrounding dead or damage tissue. Accordingly, identifying areas of dead or dying cells or tissue may be useful for some applications.

In some embodiments, aspects of the invention relate to interrogating the vibrational properties of a tissue or organ (e.g., to identify a target site for injection, for example in an infarcted heart). According to aspects of the invention, each tissue or organ has natural vibrational properties that may be altered as a result of injury or disease. Accordingly, by detecting and analyzing vibrational properties of a tissue or organ, indicia of an abnormality (e.g., associated with an injury or disease) may be detected. This information may be used to assist in detecting and/or diagnosing the injury or disease. In some embodiments, vibrational properties associated with an injury or disease may be used to identify a target tissue region and assist in the delivery of a drug, a cell preparation, or other therapy to the target tissue region.

In some embodiments, vibrations of a tissue may result from the tissue response to forces such as blood flow, air flow, etc., or any combination thereof. In some embodiments, physiological forces in a subject may cause natural vibrations of tissue or organ structures in the body. In some embodiments, organs grown ex vivo (e.g., in a bioreactor) may vibrate naturally in response to mechanical forces associated with growth in the bioreactor (e.g., fluid pumped through a vasculature, or gas pumped in and out of airways, etc.).

Natural vibrations may be detected using any suitable technique, including for example, optical techniques. In some embodiments, a laser may be used to interrogate a target region on a tissue or organ and the reflected wave energy may be evaluated to determine the vibration properties of that region. In some embodiments, the surface properties of an organ or other tissue may be evaluated. However, in some embodiments, internal properties of an organ or other tissue also may be evaluated by selecting an interrogating laser frequency and/or energy that is sufficient to penetrate to a depth of interest and provide a reflected signal that can be evaluated. For example, wavelengths from 600 to 3000 nm may be used in the IR range. These wavelengths maybe used to detect surface movement or vibrations by measuring the vibrations deflection by the response of the reflected light. In some embodiments, physical or heat vibrations may indicate vibrational patterns. In some embodiments, visible light may be used if the subject tissue is exposed. In some embodiments, IR may be used for exposed tissue and/or through tissue to make non-invasive measurements.

It should be appreciated that the resolution of the analysis may be determined by the wavelength of the interrogating laser. In some embodiments, a millimeter scale resolution may be used. However, a centimeter scale resolution also may be used since changes in vibration properties at the centimeter scale may be sufficiently informative for diagnostic and/or therapeutic applications. It should be appreciated that other resolution scales may be used as aspects of the invention are not limited in this respect.

In some embodiments, a 3-dimensional evaluation may be obtained by using a plurality of interrogating laser waves arranged in a suitable configuration. In some embodiments, an array of interrogating laser waves may be used. In some embodiments, the interrogating laser may be directed onto an organ or tissue that is surgically exposed in a subject or that is grown in a bioreactor. However, in some embodiments, an energy transfer device (e.g., an optical port) as described herein may be used in order to transmit the interrogating laser and/or receive the resulting signal. In some embodiments, a plurality of laser-transparent members may be arranged in an array on a single support member of an energy transfer device and/or a plurality of energy transfer devices may be used in order to obtain 3-dimensional information from a target organ or tissue region of interest.

It should be appreciated that the results of the analysis (e.g., the vibrational properties or the elasticity of the tissue or organ) may be displayed using any suitable technique. In some embodiments, different thresholds may be set and different levels of vibration (e.g., different vibration amplitudes) may be represented using different colors and/or intensities. In some embodiments, the vibration display may be overlaid with one or more different displays (e.g., visual images, reconstructed images, heat profiles, etc., or any combination thereof) to provide additional functionality or information. In some embodiments, certain combinations of vibration and other properties (e.g., heat) may be used for diagnostic purposes. For example, an abnormal vibration profile in combination with an abnormal heat profile may identify a organ or tissue region as diseased or injured with greater statistical significance than either profile alone.

In some embodiments, a vibration display may be overlaid with a visual display of an organ to assist in a surgical procedure. For example, a display of abnormal vibration in an infarcted heart may be overlaid with a display of the heart in order to target a therapy (e.g., a cellular injection, for example, using a stem cell or other multipotent cell preparation) to one or more damaged regions of the heart that are abnormal due to dead or dying cells caused by insufficient oxygenation.

In some embodiments, the vibration of the organ or tissue may be observed using a head-mounted device as described herein. In some embodiments, the head-mounted device is used to detect and analyze energy that was introduced using an energy transferring device as described herein to assist in transferring an interrogating laser wave (or array of laser waves) to one or more regions of a target tissue or organ of interest.

It should be appreciated that aspects of the invention may be used in combination with any suitable surgical procedure or intervention where target tissue may be identified based on abnormal vibration, heat, or other profiles, or any combination thereof. In some embodiments, a needle or surgical instrument of interest may be directly observed or may include a tag (e.g., an RFID or other suitable tag) that allows the instrument (or the operating end of the instrument) to be precisely located on the image display (e.g., on the overlay of the vibration profile, visual image, and any other suitable profile such as a heat profile). This allows the surgeon to target an injector tip (e.g., needle) or other surgical tool to a precise tissue area that was identified as damaged based on an abnormal vibration profile, heat profile, other physical profile, or a combination of two or more thereof. It should be appreciated that in some embodiments, the temperature of the instrument (or at least the working end of the instrument) may be used to detect the working end (e.g., if the temperature is higher or lower than the temperature at the site where the instrument is used). In some embodiments, an infrared detector may be used to detect the instrument, or at least a working end of the instrument. However, it should be appreciated that an infrared detector may be able to detect a working end of an instrument regardless of whether there is a temperature difference, because the infrared detector can detect differences in emissivity in addition to temperature differences.

In some embodiments, an abnormal organ or portion thereof may be replaced using a substitute organ or portion thereof that was grown in a bioreactor. Aspects of the invention may be used to assist in the transplantation or implantation procedure to identify the appropriate target regions in a recipient patient.

In some embodiments, an overlay of a vibration profile and a visual display of a region of interest may be used directly for diagnostic purposes and/or therapeutic intervention. However, in certain embodiments, a region of abnormal vibration may be identified and located in a tissue or organ using a standard reference frame (e.g., having i) a standard origin relative to defined structural properties of the tissue or organ, and ii) standard axes and units) as described herein.

In some embodiments, a normal and/or diseased profile may be defined in comparison to a known normal profile. The known normal profile may be a standard reference profile for a normal tissue or organ. In some embodiments, a subject may be scanned to obtain a personalized reference for one or more healthy organs and or tissues (provided the organs or tissues are healthy in the subject at the time of the reference analysis). This healthy reference may be stored as part of the patient medical records and used for comparison to profiles obtained during subsequent evaluations. Changes in vibration profiles, heat profiles, other physical properties, or any combination thereof, at one or more locations within a tissue or organ may be used to identify diseased regions or may be used as an initial screen to identify tissue or organs that need to be evaluated using additional techniques in order to determine their status.

In some embodiments, a normal and/or diseased profile may be defined in comparison to a known diseased profile.

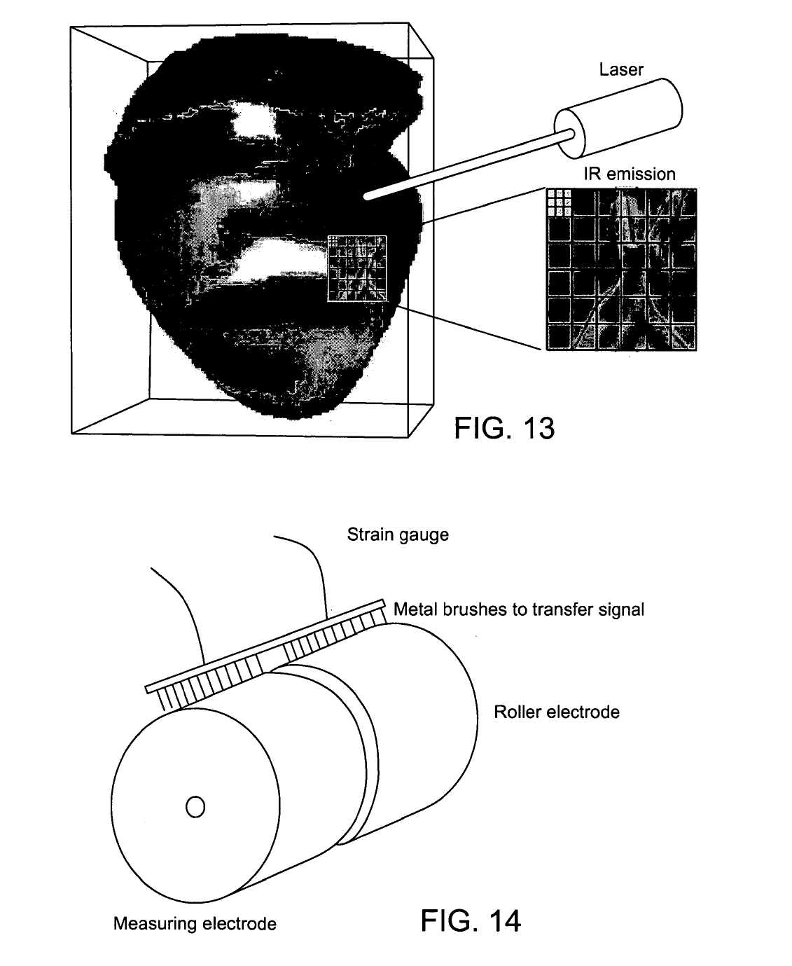

FIG. 13 illustrates a non-limiting example of a heart that is being evaluated to identify its pattern of spatial vibrational and heat distributions to determine whether normal patterns have been disrupted (which could be indicative of an infarcted heart, for example). This analysis may be performed on an organ in a patient in order to identify and/or target potential abnormalities. This analysis also may be performed on a substitute organ grown in a bioreactor to evaluate its properties and determine whether it is suitable for transplantation (e.g., by comparison to a reference substitute heart profile known to be suitable for transplantation).

In some embodiments, in addition or as an alternative to measuring natural vibration frequencies of an organ or tissue, one or more external physical and/or chemical stimuli may be applied in order to measure the vibration profile of a target region in response to the stimuli.

In some embodiments, aspects of the invention relate to methods and devices for measuring electrical signals from tissues or organs (e.g., to identify a target site for cellular injection). In some embodiments, an electrode may include a conductive rolling member at its measuring end. The rolling electrode end can be applied to the surface of a tissue or organ and is useful to measure a signal in response to pressure exerted by the rolling member on the tissue. An advantage of the rolling member is that pressure can be exerted with minimal damage to the tissue, unlike a standard electrode that includes one or more sharp tips. The applied pressure can be used to provide and maintain a good electrical contact between the tissue and the electrode and/or to physically stimulate tissue or organ surface and measure the response to the stimulus. The rolling member may be a cylinder, ball, sphere, ovoid, or other shape that can be rolled across the surface of a tissue or organ. FIG. 14 illustrates a non-limiting example of a cylindrical rolling member. An axis around which the rolling member rotates may be connected to a support structure on the electrode. However, any suitable configuration for providing a rolling tip may be used. In some embodiments, the rolling member may rotate around 2 or more axes to provide greater freedom of movement in operation. Electrical contact between the rolling member and the remainder of the electrode may be maintained using one or more metal brushes as illustrated in FIG. 14. However, it should be appreciated that other electrical connections may be used as aspects of the invention are not limited in this respect. In some embodiments, the electrode also includes a strain gauge to measure the force exerted by the electrode on to the surface of the tissue or organ. In some embodiments, the strain gauge may be connected to a controller that regulates the amount of pressure that the electrode exerts on the surface.

It should be appreciated that the rolling member includes conductive material (e.g., a metal, conductive ceramic, glass, conductive polymer, etc., or any combination thereof) on its surface. In some embodiments, the conductive surface material is supported by a non-conductive material to prevent any loss of current through the support and/or through the connections to the one or more axes. This can be useful to maximize the current that is detected through the brushes or other electrical connector.

In some embodiments, the rolling member is connected to an electrode arm that may be connected to one or more robotic motors that control the motion of the electrode on the tissue. However, in some embodiments, a hand-held measuring electrode including a rolling member may be used.

It should be appreciated that an electrode may include an array of rolling members, all of which may be connected to the same processor and/or display unit to analyze and/or represent the electrical signals measured by the rolling member(s) in any suitable format. In some embodiments, only abnormal signals are displayed.

In some embodiments, a representation of the electrical profile of an organ or tissue surface may be overlaid in a display (e.g., a head-mounted display) along with a visual display and/or one or more of a heat profile (e.g., IR profile), vibration profile, and/or other physical profile as described herein. Accordingly, electrical profiles obtained from one or more electrodes described herein may be used to monitor or target a surgical intervention as described herein in connection with other information.

In some embodiments, probes may include pressure sensors. In some embodiments, elasticity and pressure waves may be sensed through and on a surface (e.g., of a tissue or organ). In some embodiments, a probe also may have a light sensor (e.g., to detect light in the IR range, for example, from 600-3000 nm). In some embodiments, a probe may be able to detect or include filters that are adapted for oxygen-sensing (e.g., wavelength around 500 nm) or for non-oxygen-sensing (e.g., wavelength around 700 nm).

Although non-invasive imaging techniques may be used to evaluate signals from cells and tissues, tracers or markers may be used in some embodiments. For example, tracers or markers (e.g., clinically approved ones) may be used to mark a location or identify cells or for other purposes (e.g., to match sponsor and donor cells and tissues, to evaluate the physiological activity or state of the cells, etc., or any combination thereof). It should be appreciated that the tracers or markers may be tracked using chemical, electrical, spectrometric, physical (e.g., tissue pressure, temperature, size, etc., or any combination thereof) properties of the tracers or markers or of the cells or tissues associated with the tracers or markers.

In some embodiments, the radiation or other information detected from a plurality of portions or locations within a tissue or organ may be used to form a two-dimensional or three-dimensional map. The map may include, for example, a standard reference frame including one or more reference points (or reference lines). The reference point(s) or line(s) may correlate with, for example, a specific, identifiable portion of the tissue or organ of interest. For example, for a brain, skull landmarks such as bregma, lambda, and the interaural line, are commonly used as the origins of a coordinate system. Similar landmarks may be identified with the tissue or organ of interest to form one or more reference points (or lines) to generate a standard reference frame which may be specific to the type, age, and/or organism inhabiting the tissue or organ of interest. The map may also include coordinates that can allow determination of locations of each of the different portions of the tissue or organ on the map. The standard reference frame may be displayed along with the one or more images described herein (e.g., superimposed images).

It should be appreciated that the images and/or standard reference frame may be displayed using any suitable technique. In some embodiments, different thresholds may be set and different levels of the parameter being measured may be represented using different colors and/or intensities. In some embodiments, the images may be superimposed with one or more different images (e.g., images described herein such as visual images, reconstructed images, heat profiles, etc., or any combination thereof) to provide additional functionality or information. In some embodiments, certain combinations of infrared emission and other properties described herein may be used for diagnostic purposes. For example, an abnormal infrared radiation profile in combination with an abnormal heat profile may identify a organ or tissue region as diseased or injured with greater statistical significance than either profile alone.

One or more images may be displayed on any suitable display unit. In some cases, one or more images is displayed on a head-mounted display unit, an orthogonal view display unit, a cathode ray tube unit, an autostereoscopic display unit, a volumetric display unit, or a liquid crystal display unit. The image(s) displayed may be, for example, an orthogonal projection, e.g., using the data generated as described herein.

### Use of a Head-Mounted Device for Injection:

In some embodiments, aspects of the invention relate to a head-mounted device for displaying images, data, and/or other observable features of the tissue or organ of interest. The head-mounted device may include one or more of the features described above and herein. For example, in one particular embodiment, the head-mounted device may include a strap, two displays, one or more detectors (e.g., cameras or other detectors) connected to each display, and other components. A first detector may be operatively associated with a right display and a second detector may be operatively associated with a left display (e.g., one for each eye). It should be understood, however, that other configurations are possible.

In some embodiments, a head-mounted device includes a display that can be used to overlay or superimpose information (e.g., images) from different detectors. In some embodiments, two of more of the following types of information can be overlaid: a visual image, an infrared image, a Raman image, a pressure image, a temperature image, a vibrational analysis image, a fluorescence image, an image associated with emission from a non-visible contrast agent, an image from electrical analysis, and/or additional information. Such and other images may be overlaid in real-time. Additionally or alternatively, one or more images may be superimposed with one or more images that were taken of the tissue or organ of interest at an early point in time. Such images include, for example, an ultrasound image, an X-ray image, a MRI image, a CAT scan image, a positron emission tomography image, and/or a single photon emission computer tomography image. The data or images can be superimposed into a single image, or into multiple images, the specific combination of which may be chosen by the user.

In some embodiments, a head-mounted device may include two or more displays to provide a stereo image to the user. Each display may overlay two or more types of information as described above.

As described herein, different numbers and types of detectors may be operatively associated with the head-mounted device. Thus, the detecting and displaying steps described above and herein can be performed with the head-mounted device. In some embodiments, the detecting, displaying, as well as analyzing steps can all be performed with the same head-mounted device. In some instances, the head-mounted device includes two ore more detectors that allows an orthogonal viewing ability.

In some cases, a detector (e.g., a camera, such as a video camera) has an auto-focus ability (e.g., a depth perception auto-focus ability) with a sufficient dynamic range to allow the user to move his/her head and detect magnified information from the tissue, but also observe surrounding material and areas with lower magnification. The auto-focus ability may be performed in real-time. For example, the head-mounted device may be a what-you-see-is-what-you-get (WYSIWYG) optical viewing system. This can allow the user to operate other tools, whether they be surgical instruments, controller, or physical observations of other displays (e.g., monitors) or other parts of a patient being operated on or instrument being used. Certain detectors known in the art which may provide dynamic range and auto-focus ability may be used in embodiments described herein.

As noted above, the head-mounted device may include a microscope or other suitable magnification unit. The device may have, for example, at least a 10×, at least a 15×, at least a 20×, at least a 50×, at least a 100×, at least a 250×, or at least a 500× magnification ability. In some embodiments, a device can be used to monitor an event within a cell of the at least one tissue or organ of interest. In some embodiments, this magnification ability can allow the device to be used for applications such as monitoring a binding event within a cell of the at least one tissue or organ of interest. In some cases, it can be used to monitor events within a plurality of cells of at least one tissue or organ of interest.

In some embodiments, the head-mounted device comprises a binocular telescope. The device may have, for example, at least a 10×, at least a 15×, at least a 20×, at least a 50×, at least a 100×, at least a 250×, or at least a 500× reduction (e.g., demagnification) ability. In certain embodiments, the device comprises both a microscope and binocular telescope.

The head-mounted device may have other characteristics described herein, such as a source of radiation (e.g., infrared, ultraviolet, and/or other radiation described herein) such that radiation to at least one portion of the tissue or organ is emitted from the device. The device may also include a spectral filtering ability as described herein.

It should be appreciated that a head-mounted device may be powered using any suitable power source (e.g., one or more batteries, a wired connection to a power source, etc., or any combination thereof). It should be appreciated that any suitable power source, e.g., providing alternative and/or direct current, may be used.

In some embodiments, the head-mounted device includes a controller (e.g., a computer) and/or software, which may be incorporated into the device. In some embodiments, the head-mounted device may be controlled by a remote computer and information may be transmitted via a wire or wirelessly.

In some embodiments, aspects of the head-mounted device may be user-controlled. Controls may be operated using any suitable technique. In some embodiments, controls may be mounted on the device, allowing the operator to control with one or both hands. In some embodiments, controls may be voice-activated. In some embodiments, controls may be hand-held and/or foot-operated. Hand or foot controls may relay a signal to the head-mounted device via a wire, wirelessly, or a combination thereof for different functions being controlled. In some embodiments, controls may be located at a remote position and operated by a second individual who communicate with the user of the head-mounted device. The head-mounted device may also include an image stabilization control ability.

In one example, a control (such as a foot-operated control) allows a user to alter one or more parameters such as the magnification, which information to overlay (e.g., infrared, visible, vibrational, temperature, pressure, fluorescence, electrical, or other information described herein), while having free hands to operate other devices or instruments or to operate on a patient (e.g., within the body of the patient). In one embodiment, the user (e.g., a surgeon) may focus on the tissue or organ of interest and determine that an infrared emission would be helpful in determining the location of veins and arteries in an organ of interest (e.g., since the veins and arteries may have specific infrared profiles that show decreased or increased emission compared to other parts of the organ). The user could choose all or portions of the organ for targeting the detection of infrared emission data. The data can be analyzed using software within the head-mounted device, and the data generated into a two- or three-dimension image in a viewing display. At the same time, visible radiation can be detected, showing normal viewing of the organ. This data can be optionally analyzed, and then generated into a two- or three-dimensional image. If desired, the infrared and visible radiation images can be superimposed into a single image, which can allow the user to see locations of structures (e.g., veins and arteries) that the user could not have easily seen by emission in the visible spectrum. In some cases, the superimposed image can be viewed in real-time, and any adjustments by the user can be seen in the superimposed image. For example, the user could control the magnification of the superimposed image to focus in on certain portions of the organ of interest during an operation. The overlay of information can be used to identify areas for surgical intervention based on a combination of types of information. When the user looks away from the organ of interest, e.g., to obtain tools or other components for the operation, the auto-focus ability of the device may allow for instantaneous change in depth perception. Other modes of operation are also possible and envisioned within the context of the invention.

Accordingly, the head-mounted device may be used in a variety of different applications. In some embodiments, the device is adapted and arranged to be worn by a surgeon, who can use the device to perform surgery (e.g., heart surgery, incisions, injections, sutures, detectors, and/or any other interventions where enhance observations are useful, or surgery on other organs described herein). In other embodiments, the device is adapted and arranged to be worn by a phlebotomist, who can use the device to collect blood from a patient comprising the tissue or organ of interest. In yet other embodiments, the device is adapted and arranged to be worn by a dentist. Other examples of non-limiting applications where the head-mounted device can be used include animal research, clinical surgery (e.g., operating room loop replacement and surgical microscope replacement), industrial quality control (e.g., real-time product quality control packaging inspection), low vision conditions (e.g., to enhance vision), medical applications (e.g., skin and throat visualization, detection of skin lesions), process control quality control (e.g., pipe or weld inspection, circuit inspection, regenerative organs, tissue engineering, detection/identification of the chemical makeup of surfaces or contaminants in a container), security/forensics/armed forces (e.g., crime scene investigation, factory surveillance), semiconductor industry (e.g., silicon inspection, board quality control), sports (e.g., sports games).

As described herein, the head-mounted device may used to enhanced images: not only visual but combine visual images with other types of information. In some cases this provides a simple enhancement to allow a user to identify features that are not visually observable (e.g., heat profiles, vibration profiles, etc.). This allows a user to determine areas of diseased or otherwise abnormal tissue for any suitable application (e.g., for a surgical intervention). In some embodiments, enhanced images may be provided by algorithms that combine different types of information and provide new signals based on combinations of features that are shown to be clinically or physiologically relevant where any one of the individual types of information would not be sufficient. The novel information could be displayed in any fashion. For example, different colors could be used to display different properties of tissue (for example, a combination of information that is normal may be displayed in a first color, for example green, whereas a combination of information that is below or above a threshold for an abnormal tissue may be displayed in a second color, for example red). It should be appreciated that additional thresholds and/or alternative information may be provided using additional or alternative colors.

The head-mounted device may have one or more of the following benefits: a lower cost versus higher capability than traditional stereo bench scopes; a greater depth of field than regular optics since close proximity to the object is not required to have a large magnification factor and the view can be changed infinitely; the viewing angles can change with head movement so that no sophisticated stage or balancing hardware required; a small, light for portability and long-term use; it can record what is seen; it can display and record simultaneously, e.g., for teaching, mimicking SOP's; it can have multiple modes (e.g., negative, black and white, color, infrared, ultraviolet, temperature); and it can be battery or wall powered allowing for remote viewing.

### Energy Transfer Through an Energy Port:

In some embodiments, aspects of the invention relate to a “port” that can be used for enhancing observations from within a body and/or for enhancing the transmission of energy into the body. In some embodiments, a port of the invention provides a window into a patient that allows for enhanced observation and detection of physical properties of internal organs or tissues with minimal invasiveness. Such devices can be useful to assist in disease diagnosis, organ evaluation, surgical intervention, and for other medical applications.

In some embodiments, the port is an energy transfer device that promotes energy transfer into or out of a tissue, organ, or body. The skin of a body or the outer surface of a tissue or organ can impede, reflect, refract, disperse, or otherwise reduce the transfer of energy into or out of the body, tissue, or organ. This makes it more difficult to stimulate a target region in a tissue or organ non-invasively (e.g., from outside the body of a patient). This also makes it more difficult to detect energy (e.g., heat, or other energy) from within a target region in a tissue or organ non-invasively (e.g., from outside the body of a patient).

In some embodiments, a minimally invasive device may be used to help transfer energy across the skin or a surface region of a tissue or organ. In some embodiments, a minimally invasive device includes an insertable member that can be inserted into or through the skin or surface region to provide a pathway for energy transfer without requiring an invasive surgical procedure. In some embodiments, it is sufficient to insert the member to a minimal depth (e.g., a few mm across the skin) that provides for enhanced energy transfer to allow evaluation of surrounding or underlying tissue or organ structures without cutting into the tissue or organ structures. The insertable member may be elongated so that it can penetrate to a desired depth of the skin or surface region while one end remains exposed (e.g., protruding on the outside of the skin or surface) and can be connected to an energy source and/or detector.

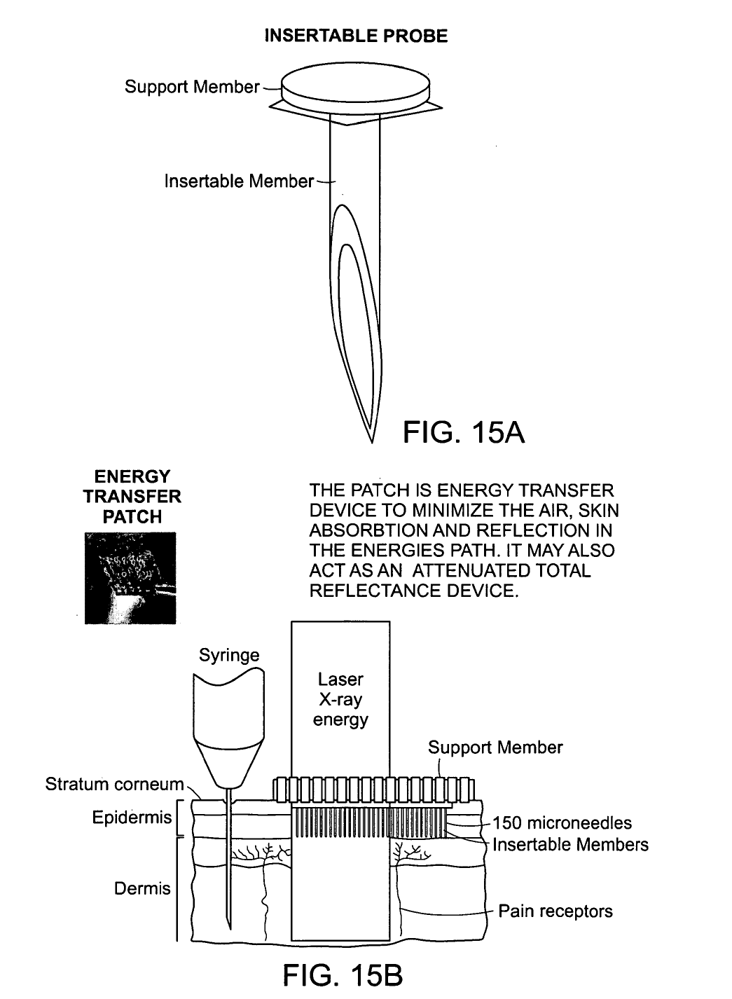

FIG. 15A shows a non-limiting example of an insertable probe comprising an elongated insertable member attached to a support member. In use, the insertable member can be inserted through the skin whereas the support member is not inserted. The support member remains at the surface of the skin or other tissue or organ surface. In some embodiments, the support member is connected to an energy source and energy can be transferred via the support member to the inserted elongated member and from there to the organ or tissue on the other side of the skin or other surface. In certain embodiments, the support member is connected to an energy detector and energy from within an organ or tissue can be transferred via the inserted elongated member to the support member from where it is transferred to the detector.

In FIG. 15A, the elongated member is shown as a hollow cylinder with an outer layer and an inner volume. It should be appreciated that air or fluid in the inner volume can transfer energy, for example light. In some embodiments, the inner volume may be filled with a material that promotes the transfer of a particular energy (e.g., wavelengths from 340 nm to 3000 nm). The material may be any suitable material, for example, one of the following non-limiting materials: glasses, silicon, polymers that have transmission windows in analysis areas of interest, etc., or any combination thereof. In some embodiments, optical pipes may be used to bring light in and/or collect reflected light. In some embodiments, a material may be an IR conducting material such as silicon or polymers that can be used in an attenuated total reflectance mode, or plastic, or any combination thereof.

The following tables indicate wavelengths of interest for different types of bonds that may be considered for cell, tissue, and/or organ evaluation according to aspects of the invention.

| | Type of | Specific type of | | | |
|---|---|---|---|---|---|
| Bond | bond | bond | Absorption peak | | Appearance |
| C—H | Alkyl | Methyl | 1260 | cm⁻¹ | strong |
| | | | 1380 | cm⁻¹ | weak |
| | | | 2870 | cm⁻¹ | medium to strong |
| | | | 2960 | cm⁻¹ | medium to strong |
| | | methylene | 1470 | cm⁻¹ | strong |
| | | | 2850 | cm⁻¹ | medium to strong |
| | | | 2925 | cm⁻¹ | medium to strong |
| | | methine | 2890 | cm⁻¹ | weak |
| | vinyl | C═CH₂ | 900 | cm⁻¹ | strong |
| | | | 2975 | cm⁻¹ | medium |
| | | | 3080 | cm⁻¹ | medium |
| | | C═CH | 3020 | cm⁻¹ | medium |
| | | monosubstituted | 900 | cm⁻¹ | strong |
| | | alkenes | 990 | cm⁻¹ | strong |
| | | cis-disubstituted | 670-700 | cm⁻¹ | strong |
| | | alkenes | | | |
| | | trans-disubstituted | 965 | cm⁻¹ | strong |
| | | alkenes | | | |
| | | trisubstituted | 800-840 | cm⁻¹ | strong to medium |
| | | alkenes | | | |
| | aromatic | benzene/sub. | 3070 | cm⁻¹ | weak |
| | | benzene | | | |
| | | monosubstituted | 700-750 | cm⁻¹ | strong |
| | | benzene | 690-710 | cm⁻¹ | strong |
| | | ortho-disub. | 750 | cm⁻¹ | strong |
| | | benzene | | | |
| | | meta-disub. | 750-800 | cm⁻¹ | strong |
| | | benzene | 860-900 | cm⁻¹ | strong |
| | | para-disub. | 800-860 | cm⁻¹ | strong |
| | | benzene | | | |
| | alkynes | any | 3300 | cm⁻¹ | medium |
| | aldehydes | any | 2720 | cm⁻¹ | medium |
| | | | 2820 | cm⁻¹ | |
| C—C | acyclic | monosub. alkenes | 1645 | cm⁻¹ | medium |
| | C—C | 1,1-disub. alkenes | 1655 | cm⁻¹ | medium |
| | | cis-1,2-disub. | 1660 | cm⁻¹ | medium |
| | | alkenes | | | |
| | | trans-1,2-disub. | 1675 | cm⁻¹ | medium |
| | | alkenes | | | |
| | | trisub., tetrasub. | 1670 | cm⁻¹ | weak |
| | | alkenes | | | |
| | conjugated | dienes | 1600 | cm⁻¹ | strong |
| | C—C | | 1650 | cm⁻¹ | strong |
| | with | | 1625 | cm⁻¹ | strong |
| | benzene | | | | |
| | ring | | | | |
| | with C═O | | 1600 | cm⁻¹ | strong |
| | C═C | any | 1640-1680 | cm⁻¹ | medium |
| | (both sp²) | | | | |
| | aromatic | any | 1450 | cm⁻¹ | weak to strong |
| | C═C | | 1500 | cm⁻¹ | (usually 3 or 4) |
| | | | 1580 | cm⁻¹ | |
| | | | 1600 | cm⁻¹ | |
| | C≡C | terminal alkynes | 2100-2140 | cm⁻¹ | weak |
| | | disubst. alkynes | 2190-2260 | cm⁻¹ | very weak (often |
| | | | | | indisinguishable) |
| C═O | Aldehyde/ | saturated | 1720 | cm⁻¹ | |
| | | aliph./cyclic 6- | | | |
| | ketone | membered | | | |
| | | α,β-unsaturated | 1685 | cm⁻¹ | |
| | | aromatic ketones | 1685 | cm⁻¹ | |
| | | cyclic 5- | 1750 | cm⁻¹ | |
| | | membered | | | |
| | | cyclic 4- | 1775 | cm⁻¹ | |
| | | membered | | | |
| | | aldehydes | 1725 | cm⁻¹ | influence of |
| | | | | | conjugation (as with |
| | | | | | ketones) |
| | carboxylic | saturated | 1710 | cm⁻¹ | |
| | acids/derivates | carboxylic acids | | | |
| | | unsat./aromatic | 1680-1690 | cm⁻¹ | |
| | | carb. acids | | | |
| | | esters and lactones | 1735 | cm⁻¹ | influenced by |
| | | | | | conjugation and ring |
| | | | | | size (as with ketones) |
| | | anhydrides | 1760 | cm⁻¹ | |
| | | | 1820 | cm⁻¹ | |
| | | acyl halides | 1800 | cm⁻¹ | |
| | | amides | 1650 | cm⁻¹ | associated amides |
| | | carboxylates | 1550-1610 | cm⁻¹ | |
| | | (salts) | | | |
| | | amino acid | 1550-1610 | cm⁻¹ | |
| | | zwitterions | | | |
| O—H | alcohols, | low concentration | 3610-3670 | cm⁻¹ | |
| | phenols | high concentration | 3200-3400 | cm⁻¹ | broad |
| | carboxylic | low concentration | 3500-3560 | cm⁻¹ | |
| | acids | high concentration | 3000 | cm⁻¹ | broad |
| N—H | primary | any | 3400-3500 | cm⁻¹ | strong |
| | amines | | 1560-1640 | cm⁻¹ | strong |
| | secondary | any | >3000 | cm⁻¹ | weak to medium |
| | amines | | | | |
| | ammonium | any | 2400-3200 | cm⁻¹ | multiple broad peaks |
| | ions | | | | |
| C—O | alcohols | primary | 1040-1060 | cm⁻¹ | strong, broad |
| | | secondary | ~1100 | cm⁻¹ | strong |
| | | tertiary | 1150-1200 | cm⁻¹ | medium |
| | Phenols | any | 1200 | cm⁻¹ | |
| | Ethers | aliphatic | 1120 | cm⁻¹ | |
| | | aromatic | 1220-1260 | cm⁻¹ | |
| | carboxylic | any | 1250-1300 | cm⁻¹ | |
| | acids | | | | |
| | Esters | any | 1100-1300 | cm⁻¹ | two bands (distinct |
| | | | | | from ketones, which |
| | | | | | do not possess a C—O |
| | | | | | bond) |
| C—N | aliphatic | any | 1020-1220 | cm⁻¹ | often overlapped |
| | amines | | | | |
| | C═N | any | 1615-1700 | cm⁻¹ | similar conjugation |
| | | | | | effects to C═O |
| | C≡N | unconjugated | 2250 | cm⁻¹ | medium |
| | (nitriles) | conjugated | 2230 | cm⁻¹ | medium |
| | R—N—C | any | 2165-2110 | cm⁻¹ | |
| | (isocyanides) | | | | |
| | R—N═C═S | any | 2140-1990 | cm⁻¹ | |
| C—X | fluoroalkanes | ordinary | 1000-1100 | cm⁻¹ | |
| | | trifluromethyl | 1100-1200 | cm⁻¹ | two strong, broad |
| | | | | | bands |
| | chloroalk | any | 540-760 | cm⁻¹ | weak to medium |
| | [symbol] s | | | | |
| | bromoalkanes | any | 500-600 | cm⁻¹ | medium to strong |
| | iodoalkanes | any | 500 | cm⁻¹ | medium to strong |
| N—O | nitro | aliphatic | 1540 | cm⁻¹ | stronger |
| | compounds | | 1380 | cm⁻¹ | weaker |
| | | aromatic | 1520, 1350 | cm⁻¹ | lower if conjugated |

[symbol]

indicates data missing or illegible when filed

| Functional | | | | | | |
|---|---|---|---|---|---|---|
| Class | Range (cm⁻¹) | Intensity | Assignment | Range (cm⁻¹) | Intensity | Assignment |
| Alkanes | 2850-3000 | str | CH₃, CH₂ & CH | 1350-1470 | med | CH₂ & CH₃ |
| | | | 2 or 3 bands | 1370-1390 | med | deformation |
| | | | | 720-725 | wk | CH₃ deformation |
| | | | | | | CH₂ rocking |
| Alkenes | 3020-3100 | med | ═C—H & ═CH₂ (usually | 880-995 | str | ═C—H & ═CH₂ |
| | | | sharp) | 780-850 | med | (out-of-plane |
| | 1630-1680 | var | C═C (symmetry reduces | | | bending) |
| | | | intensity) | 675-730 | med | cis-RCH═CHR |
| | 1900-2000 | str | C═C asymmetric stretch | | | |
| Alkynes | 3300 | str | C—H (usually sharp) | 600-700 | str | C—H |
| | 2100-2250 | var | C≡C (symmetry reduces | | | deformation |
| | | | intensity) | | | |
| Arenes | 3030 | var | C—H (may be several | 690-900 | str-med | C—H bending & |
| | | | bands) | | | ring puckering |
| | 1600 & | med- | C═C (in ring) (2 bands) | | | |
| | 1500 | wk | (3 if conjugated) | | | |
| Alcohols & | 3580-3650 | var | O—H (free), usually sharp | 1330-1430 | med | O—H bending |
| Phenols | 3200-3550 | str | O—H (H-bonded), usually | | | (in-plane) |
| | | | broad | 650-770 | var-wk | O—H bend (out- |
| | 970-1250 | str | C—O | | | of-plane) |
| Amines | 3400-3500 | wk | N—H (1°-amines), 2 bands | 1550-1650 | med-str | NH₂ scissoring |
| | (dil. soln.) | | | | | (1°-amines) |
| | 3300-3400 | wk | N—H (2°-amines) | 660-900 | var | NH₂ & N—H |
| | (dil. soln.) | | | | | wagging |
| | 1000-1250 | med | C—N | | | (shifts on H- |
| | | | | | | bonding) |
| Aldehydes & | 2690-2840 | med | C—H (aldehyde C—H) | 1350-1360 | str | α-CH₃ bending |
| Ketones | (2 [symbol] | | | 1400-1450 | str | α-CH₂ bending |
| | bands) [symbol] | | | 1100 | med | C—C—C bending |
| | 1720-1740 [symbol] | str | C═O (saturated aldehyde) | | | |
| | 1710-1720 [symbol] | str | C═O (saturated ketone) | | | |
| | 1690 [symbol] | str | aryl ketone | | | |
| | 1675 [symbol] | str | α,β-unsaturation | | | |
| | 1745 [symbol] | str | cyclopentanone | | | |
| | 1780 [symbol] | str | cyclobutanone | | | |
| Carboxylic Acids | 2500-3300 [symbol] | str | O—H (very broad) | 1395-1440 | med | C—O—H bending |
| & Derivatives | (acids) [symbol] | | | 1590-1650 | med | N—H (1ᵢ-amide) II |
| | overlap C—H [symbol] | str | C═O (H-bonded) | | | band |
| | 1705-1720 [symbol] | | | 1500-1560 | med | N—H (2ᵢ-amide) II |
| | (acids) [symbol] | | | | | band |
| | 1210-1320 [symbol] | med- | O—C (sometimes 2-peaks) | | | |
| | (acids) [symbol] | str | | | | |
| | 1785-1815 [symbol] | str | C═O | | | |
| | (acyl [symbol] | | | | | |
| | halides) [symbol] | | | | | |
| | 1750 & [symbol] | str | C═O (2-bands) | | | |
| | 1820 [symbol] | | | | | |
| | (anhydrides) [symbol] | | | | | |
| | 1040-1100 [symbol] | str | O—C | | | |
| | 1735-1750 [symbol] | str | C═O | | | |
| | (esters) [symbol] | | | | | |
| | 1000-1300 [symbol] | str | O—C (2-bands) | | | |
| | 1630-1695 [symbol] | str | C═O (amide I band) | | | |
| | (amides) [symbol] | | | | | |
| Nitriles | 2240-2260 [symbol] | Med | C≡N (sharp) | | | |
| Isocyanates, | 2100-2270 [symbol] | med | —N═C═O, —N═C═S | | | |
| Isothiocyanates, | | | N═C═N—, —N₃, C═C═O | | | |
| Diimides, Azides | | | | | | |
| & Ketenes | | | | | | |

[symbol]

indicates data missing or illegible when filed

In some embodiments, the inner volume serves as a conduit that houses one or more energy transfer fibers (e.g., optical fibers).

In some embodiments, the inserted end of the elongated member is open as shown in FIG. 15A. However, in some embodiments the inserted end may be closed. A closed end may have an energy transferring window that allows energy to pass from the device into the tissue or organ or vice-versa. In some embodiments, the entire inserted member is constructed of energy-transferring material. However, in some embodiments, a closed end of the insertable member may allow more energy to be transferred (e.g., it may be more transparent) than the walls of the insertable member. Accordingly, the end may be a “window” that allows energy (e.g., light) through.

In some embodiments, the insertable member is made entirely of an energy transferring (e.g., light transparent) material without a separate outer wall. In some embodiments, the insertable member may be an optical fiber or a fiber optic bundle. In some embodiments, the insertable may include (e.g., within an outer wall) or consist entirely of any one or more of the following non-limiting materials: glass, polymers, silver halide, chacalgonite or a polymer with a absorption window in the area of interest, etc., or any combination thereof.

It should be appreciated that the inserted end of the insertable member may be shaped to promote insertion into tissue. For example, it may be tapered, pointed, or otherwise sharpened and/or sufficiently rigid to assist or promote insertion (e.g., through skin). However, in some embodiments, the diameter of the elongated insertable member may be sufficiently small to allow easy insertion without requiring any particular shape at the tip. However, regardless of the size of the insertable member, it may have any shape at the inserted tip (e.g., a single point, multiple points, serrated edges, smooth edges, regular or regular shapes, or any combination thereof).

FIG. 15A shows the elongated member as cylindrical in shape. However, it should be appreciated that the elongated member may have any cross-sectional shape, regardless of whether it is hollow or not. For example, the cross-section of the elongated member may be a triangle, a square, a rectangle, or other parallelogram, a circle, an oval, a pentagon, a hexagon, or have any number of sides that may be straight, curved, or a combination thereof, as aspects of the invention are not limited in this respect. Accordingly, a cross-section of the elongated member may be regular or irregular in shape.

FIG. 15A shows the elongated member as being straight along its length. However, it should be appreciated that it may be curved, tapered, flared, or any combination thereof. In some embodiments, one or more constricted and/or expanded sections may be included along the length of the elongated member.

In some embodiments, the elongated member may be between about one or more microns long and one or more centimeters long. However, other lengths may be used as aspects of the invention are not limited in this respect. In some embodiments, the cross-sectional distances may be between about one or more tuns and about one or ore mms across. However, other lengths may be used as aspects of the invention are not limited in this respect.

Regardless of shape or size and whether it is constructed of a single material or two or more different materials, one or more portions of the insertable member may be coated (e.g., with a protective coating, for example, to prevent corrosion or degradation). In some embodiments the insertable tip of the member may be coated. In some embodiments, the coating may provide additional structural properties to prevent bending or other deformation in use.

It should be appreciated that the insertable member may be provided alone without the support member. However, in some embodiments, two or more insertable members may be connected to a single support member. Accordingly, a device of the invention may include a linear or two-dimensional array of two or more insertable members.

FIG. 15B shows a non-limiting embodiment of a device having an array of insertable members attached to a first surface of a support member thereby forming a patch that can be applied to the skin of a subject (or the surface of an organ or tissue). Upon application, the insertable members penetrate the skin (or surface of the organ or tissue) to provide a “window” that allows energy to flow more freely in both directions across the skin (or other surface). In some embodiments, an array allows energy to be applied to a greater volume of underlying tissue than would be allowed by a single member. In some embodiments, an array allows a three-dimensional reconstruction of the energy spectrum from a volume of tissue beneath the applied device.

FIG. 15B shows a regular array. However, it should be appreciated that a plurality of insertable members may be arranged in any pattern on the support member. The pattern may have any geometry, it may be regular, irregular, etc., or any combination thereof at different locations on the surface of the support member. It also should be appreciated that in some embodiments the density of the insertable members may be constant across the surface of the support member. However, in other embodiments the density of the insertable members may vary across the surface of the support member. It also should be appreciated that a single support member may have an array of insertable members having different sizes. For example, different lengths may be adapted for providing or detecting energy at different depths in a tissue. Different cross-sectional areas may be provided for providing or detecting different types and/or levels of energy. It also should be appreciated that an array may have any suitable number of insertable members (e.g., 5-10, 10-20, 20-50, 50-100, 100-200, or other number).

FIG. 15B shows the axes of the insertable members forming a right angle with the surface plane of the support member. However, it should be appreciated that any angle may be used. In some embodiments, all of the insertable members are in parallel and their axes all form the same angle with the surface plane of the support member. However, in some embodiments the axes of different insertable members may form different angles with the surface plane of the support member.

Regardless of the number of insertable members attached to a first surface of a support member, the support member may have any suitable shape. FIG. 15 shows a square support member. However, the shape of the first surface of the support member may be a disc, a ring, an oval, a rectangle, a pentagon, a hexagon, or have any other regular or irregular shape as aspects of the invention are not limited in this respect. The thickness of the support member is generally smaller than the dimensions of the first surface area. The thickness also is generally uniform. However, support members may have any suitable thickness and the thickness may be different at different positions as aspects of the invention are not limited in this respect. Accordingly, a device may resemble a patch that has an array of sharp elements (e.g., needle-like structures) on one surface.

The support member may be made of any suitable material or combination of materials. In some embodiments, a support member is rigid. In some embodiments, a support member is flexible. A support member may be shaped to conform to the overall surface shape and/or features of a target tissue or skin. In some embodiments, a support member is essentially a single layer of material. However, in some embodiments, a support member may include two or more layers of different material.

The insertable member(s) may be made of any suitable material. In some embodiments, the material is sufficiently rigid to allow insertion into a target skin or tissue.

It should be appreciated that in some embodiments the support member and/or insertable member may independently include one or more metallic, ceramic, polymeric, glass, plastic, other material, or any combination thereof, in their structure.

It should be appreciated that in some embodiments each insertable member on a support member may be independently connected to a separate energy source and/or detector. In certain embodiments, each elongated member may be connected to the same energy source and/or detector. In yet other embodiments, two or more insertable members may be connected to different energy sources and/or detectors while at least two insertable members are connected to at least one of the energy sources and/or detectors.

It should be appreciated that the configuration of the device (including the size, pattern, density, connections to energy sources and/or detectors) may be adapted for particular uses. In some embodiments, a single device may be used for stimulation and/or detection. In some embodiments, separate devices may be used for stimulation or detection. In some applications, only a detection mode is used. In certain applications, only the stimulation mode is used. However, both may be used as aspects of the invention are not limited in this respect.

In some embodiments, a first subset of the insertable members on an array are configured for detection whereas a second subset of the insertable members is configured for energy transduction (e.g., stimulation). For example, the first subset may be connected to one or more detectors, whereas the second subset may be connected to an energy generator.

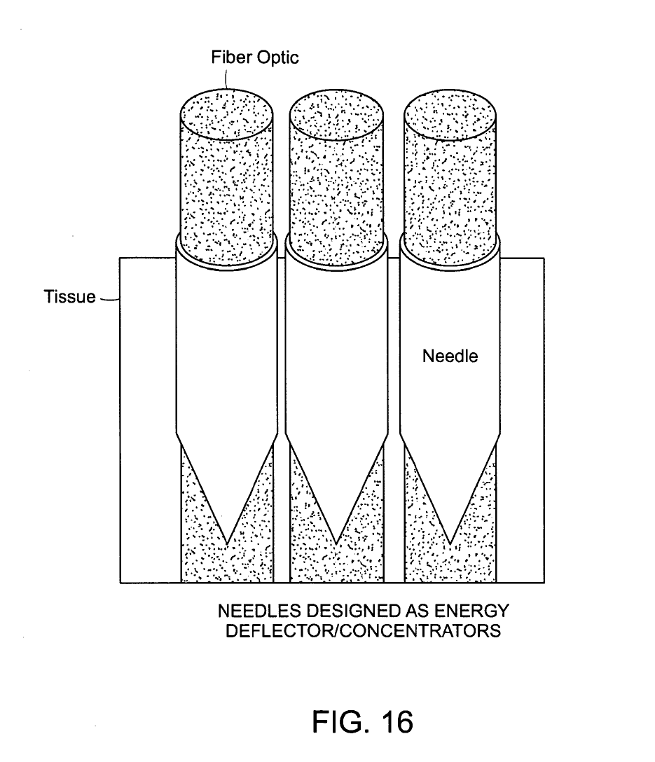

It should be appreciated that the term “promote” as used herein in the context of material that promotes energy transfer can refer to material that allows energy to be transferred without attenuation or dispersion or any other form of signal reduction (or with reduced attenuation, dispersion, or other form of signal reduction relative to the skin or other organ or tissue material through which the energy is being transferred). For example, a material that promotes energy transfer can be a material that conducts the energy more efficiently and/or with less distortion. In some embodiments, such a material may be transparent to light (e.g., visible light or infrared light, etc., or any combination thereof). However, in some embodiments, a material that promotes energy transfer may be one that concentrates, deflects, or focuses the energy. For example, FIG. 16 illustrates an embodiment of insertable elements that are designed as energy deflectors/concentrators.

It should be appreciated that in some embodiments a device may be applied temporarily to a subject's skin or the surface of an organ or tissue of interest during a surgical intervention, a diagnostic analysis, or for other short term medical applications. After use, the device may be removed (e.g., peeled off) and the underlying surface may not need any further treatment (e.g., no sutures or other form of surgical sealing may be required). In some embodiments, the surface may be sterilized after removal of the device, but this may not be required.

In certain embodiments, however, a device may be implanted into a subject (e.g., into the skin of a subject) to provide a permanent port that can be used to stimulate and or evaluate underlying body regions as described herein.

Devices described herein may be connected to an energy source and/or detector using any suitable structures. In some embodiments, optical fibers may be connected to the second surface of the support member (opposite from the first surface of the support member) and also connected to an energy source and/or detector. Suitable controllers and processors may be used to regulate stimulation and/or analyze and evaluate energy transmitted via the inserted members.

Other aspects of the invention relate to energy transfer devices or material that alter the transfer of energy into or out of the body without requiring an implantable port or patch. In some embodiments, clothing, wraps, vessels, or other devices may be used to either promote or disrupt energy transfer into or out of a subject's body or organ, or a particular region thereof. For example, in some embodiments mesh clothing may be used to enhance the transfer of energy into a subject's body during an MRI or other scan. This can be useful to reduce the exposure of the subject and also may provide enhanced images. In some embodiments, in order to increase the efficiency of energy transfer in an MRI, a mesh clothing could be used on a subject with any material absorbing or reflecting as a light guide in the ports.

Aspects of the invention may be used to detect one or more different types of energy. In some embodiments energy profiles (e.g., 2 dimensional or 3 dimensional) energy profiles may be determined for target organs or tissue areas of interest. Energy profiles for different types of energy (e.g., heat profiles, vibrational property profiles) may be evaluated independently or overlaid to provide additional information. In some embodiments, one or more energy profiles may be overlaid with a visual image or representation (e.g., reconstructed model) of a target organ or tissue area of interest. It should be appreciated that information about any suitable parameter (or combination of profiles) may be used, including but not limited to the following: temperature, MRI data, Raman data, fluorescence, IR data (e.g., within the 600-3000 nm wavelength range or a subset of that range), visible data (e.g., within the 350 nm to 599 nm wavelength range, or a subset of that range).

In some embodiments, one or more therapies of the invention may be combined with a laser therapy (e.g., to treat dead or dying tissue, for example in the context of an infarct). In some embodiments, a laser therapy (e.g., using low energy laser irradiation) may be used to irradiate tissue that is exposed (e.g., during surgery) in combination with administering one or more appropriate cellular preparations. However, a laser therapy may be combined with an energy port described herein to allow the irradiation to be appropriately targeted to an infarct without requiring surgery to expose the target tissue. In some embodiments, the laser irradiation is delivered through an energy port that has been introduced at an appropriate site within a patient's body.

### Printers for Compositions Comprising Cells

In some aspects of the invention, printers are provided for printing compositions comprising biological cells. The printers may be used in any of a variety of ways to print cells. For example, cells may printed on an in vitro substrate, such as, for example, a cover slip surface, cell culture plate or well bottom, an artificial or isolated extracellular matrix, a natural or synthetic scaffold, etc. In some embodiments, cells may printed on a biological tissue, which may either be an isolated tissue or an in vivo tissue. For example, cells may be printed directly on an isolated tissue, e.g., a dermal tissue. In another example, cells may be printed directly on a wound (e.g., a burn, an ulcer, infarction, etc.) to provide cells (e.g., stem cells, skin cells, etc.) for repairing the wound.

In some embodiments, printers for printing biological cells are provided. The printer typically comprises a print head and one or more motors or devices for moving the print head to control deposition of the composition onto a substrate. The print head is typically designed and configured to translate and/or rotate along or about one or more axes. In some cases, the print head may be designed and configured to move in three-dimensional space with 1, 2, 3, 4, 5 or 6 degrees of freedom. Accordingly, the print head may be designed and configured to move forward-backward, up-down, and/or left-right (translation in three perpendicular axes). In some embodiments, the print head is designed and configured to rotate about one, two, or three perpendicular axes (i.e., pitch, yaw, roll).

Typically the print head is designed and configured to house a composition to be printed. In some embodiments, the print head comprises a removable print cartridge that houses a composition to be printed. The print head is often designed and configured to have one or more temperature control elements that heat and/or cool the composition to maintain cells at a predetermined temperature. In some embodiments, the temperature control elements include a heating and/or cooling element. In some embodiments, the temperature control element includes a thermocouple to measure the temperature in the cartridge. In some embodiments, the temperature control elements are designed and configured to maintain a temperature in a range of 0° C. to 10° C., 5° C. to 20° C., 10° C. to 40° C., 20° C. to 50° C., 4° C. to 37° C. or 0° C. to 50° C. In some embodiments, the temperature control elements are designed and configured to maintain a temperature of up to 4° C., 10° C., 20° C., 30° C., 40° C., 50° C. or more.

The print head is also typically designed and configured to maintain any of a variety of other parameters important for cell homeostasis, including, for example, O₂ saturation, pH, nutrient concentration, etc. The print head typically comprises one or more fluid conduits for adding and/or removing fluids, e.g., for adding a buffer, for perfusing a gas, e.g., CO₂, O₂, etc. The print head is also designed and configured to release the composition comprising cells onto a substrate in a controlled manner. In some embodiments, the print head controls the volume of the composition that is deposited and/or the relative location at which the composition is deposited. The print head may be fluidically connected with one or more pumps, e.g., one or more pumps that create a pressure gradient sufficient to expel the composition from the print head. In some embodiments, the print head is designed and configured to spray droplets of the composition comprising cells onto a substrate. Thus, in some embodiments, the printer functions similar to an inkjet printer that sprays droplets of ink. In some embodiments the print head has a face plate with a plurality of nozzles. In some embodiments, each nozzle has an outlet in a range of 0.05 to 200 μm in diameter, 1 to 100 μm in diameter, 5 to 200 μm in diameter, or 10 to 50 μm in diameter. A plurality of nozzles with the same or different diameters may be provided in some embodiments. Though in some embodiments the nozzles have a circular opening, other suitable shapes may be used, e.g., oval, square, rectangle, etc., taking into account the relative size of the cells intended to be deposited.

In some embodiments, a printer comprises one or more devices or components for particle filtration, O₂ adjustment, CO₂ maintenance, pH adjustment, nutritional adjustments, waste product removal, etc. In some embodiments, these devices or components are integrated into or coupled with the printer head, e.g., intergrated into a printer cartridge. In some embodiments, the printers serves as an injecting device, defrosting device, and/or cell preparation device. In some embodiments, the printers are designed and configured to maintain the metabolic, anatomical, and/or physiological integrity of cells, thus ensuring cells are viable and functionally active following printing.

In some embodiments, printers may be designed and configured to print a biopolymer or inorganic polymer to create printed organs and/or tissues. In some embodiments, printers may be designed and configured to print a combination of biological cells and a biopolymer or inorganic polymer to create printed organs and/or tissues.

Having thus described several embodiments with respect to aspects of the inventions, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A system for introducing cells into a tissue, comprising:
a working end of a cell introducing device having at least one opening for releasing cells at a tissue site.

2. A method for introducing cells into a tissue, comprising:
releasing cells from an opening of a working end of a cell introducing device at a tissue site.

3-40. (canceled)

41. The method of claim 2, wherein a cell preparation is warmed to a threshold temperature within a predetermined time period prior to injection.

42. The method of claim 41, wherein the predetermined time period is 1 second −60 minutes.

43. The method of claim 41, wherein the threshold temperature is between liquid nitrogen temperature and body temperature.

44. The method of claim 43, wherein the threshold temperature is 5-10 degrees centigrade below body temperature.

45. The method of claim 2, wherein the cells are released below or above a threshold pressure at the tissue site.

46. The method of claim 45, wherein the threshold pressure is between 100 and 200 mm mercury.

47. The method of claim 45, wherein the threshold pressure is greater or less than the blood pressure of the recipient.

48-50. (canceled)

51. The system of claim 1, further comprising a microfluidic circuit in fluid connection with the working end of the cell introducing device.

52. The system of claim 51, wherein the microfluidic circuit comprises a gas-permeable membrane, ceramic porous material, metallic fritted material.

53. The system of claim 51, wherein the microfluidic circuit comprises one or more sensors selected from the group consisting of sensors for pH, oxygen, carbon dioxide, temperature, pressure, or specific degradation products of cellular metabolism.

54. The system of claim 51, further comprising a working end that is 0.001-250 mm long.

55-57. (canceled)

58. A method for delivering infrared light into a tissue, the method comprising piercing the tissue surface using a device that has an insertable member that conducts infrared light, and transmitting infrared light through the insertable member into the tissue.

59-61. (canceled)
